SEQUENCE LISTING (65 sequences)

SEQ ID NO: 1 - protein, 9 aa, Artificial
  motif Q
GFEEPSAIQ

SEQ ID NO: 2 - protein, 8 aa, Artificial
  motif I
AQSGTGKT

SEQ ID NO: 3 - protein, 6 aa, Artificial
  motif Ia
PTRELA

SEQ ID NO: 4 - protein, 5 aa, Artificial
  motif Ib
TPGRV

SEQ ID NO: 5 - protein, 4 aa, Artificial
  motif II
DEAD

SEQ ID NO: 6 - protein, 3 aa, Artificial
  motif III
SAT

SEQ ID NO: 7 - protein, 9 aa, Artificial
  motif IV
VIFCNTRRK

SEQ ID NO: 8 - protein, 9 aa, Artificial
  motif V
TDLLARGID

SEQ ID NO: 9 - protein, 8 aa, Artificial
  motif VI
HRIGRTGR

SEQ ID NO: 10 - DNA, 1212 bp, Leishmania infantum
  CDS (1)..(1212)
atggcgcagaatgataagatcgccccccaggaccaggactccttcctcgatgaccagcccggcgttcgcccgatcccgtccttcgacgacatgccgctgcaccagaacctgctgcgcggcatctactcgtacgggttcgagaagccgtccagcatccagcagcgcgcgatagcccccttcacgcgcggcggcgacatcatcgcgcaggcccagtccggtaccggcaagacgggtgccttctccatcggtctgctgcagcgcctggacttccgccacaacctgatccagggcctcgtgctctcccccactcgcgagctggccctgcagacggcggaggtgatcagccgcatcggtgagttcctgtcgaacagctccaagttctgcgagacctttgtcggcggcacgcgcgtgcaggatgacctgcgcaagctgcaggccggcgtcatcgttgccgtgggcacgccgggccgcgtgtccgacgtgatcaagcgcggcgcgctgcgcacggagtcgctgcgcgtgctggtgctcgacgaggctgatgagatgctgtctcagggcttcgcggaccagatttacgagatcttccgcttcctgccgaaggacatccaggtcgcgctcttctccgccacgatgccggaggaggtgctggagctgacgaagaagttcatgcgcgaccccgtgcgcattctcgtgaagcgcgagagcctgacgctggagggcatcaagcagttcttcatcgccgtcgaagaggagcacaagctggacacgctgatggacctgtacgagaccgtgtccatcgcgcagtccgtcatcttcgccaacacgcgccgcaaggtggactggatcgccgagaagctgaaccagagcaaccacaccgtcagcagcatgcacgccgagatgcccaagagcgaccgcgagcgcgtcatgaacaccttccgcagcggcagctcccgcgtgctcgtcacgaccgacctcgtggcgcgcggtatcgacgtgcaccacgtgaacatcgtcatcaacttcgacctgccaacgaacaaggagaactacctgcatcgcattggccgcggcggccgctacggccgtaagggtgttgccatcaacttcgtgacggagaaggacgtggagctgctgcacgagatcgaggcgcactaccacacgcagatcgacgagctcccggtcgacttcgctgcctaccttggcgagtaa

SEQ ID NO: 11 - protein, 403 aa, Leishmania infantum
MAQNDKIAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRDPVRILVKRESLTLEGIKQFFIAVEEEHKLDTLMDLYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGRKGVAINFVTEKDVELLHEIEAHYHTQIDELPVDFAAYLGE

SEQ ID NO: 12 - protein, 395 aa, Saccharomyces cerevisiae
MSEGITDIEESQIQTNYDKVVYKFDDMELDENLLRGVFGYGFEEPSAIQQRAIMPIIEGHDVLAQAQSGTGKTGTFSIAALQRIDTSVKAPQALMLAPTRELALQIQKVVMALAFHMDIKVHACIGGTSFVEDAEGLRDAQIVVGTPGRVFDNIQRRRFRTDKIKMFILDEADEMLSSGFKEQIYQIFTLLPPTTQVVLLSATMPNDVLEVTTKFMRNPVRILVKKDELTLEGIKQFYVNVEEEEYKYECLTDLYDSISVTQAVIFCNTRRKVEELTTKLRNDKFTVSAIYSDLPQQERDTIMKEFRSGSSRILISTDLLARGIDVQQVSLVINYDLPANKENYIHRIGRGGRFGRKGVAINFVTNEDVGAMRELEKFYSTQIEELPSDIATLLN

SEQ ID NO: 13 - protein, 399 aa, Saccharomyces cerevisiae
MSFDREEDQKLKFKTSKKLKVSSTFESMNLKDDLLRGIYSYGFEAPSSIQSRAITQIISGKDVIAQAQSGTGKTATFTIGLLQAIDLRKKDLQALILSPTRELASQIGQVVKNLGDYMNVNAFAITGGKTLKDDLKKMQKHGCQAVSGTPGRVLDMIKKQMLQTRNVQMLVLDEADELLSETLGFKQQIYDIFAKLPKNCQVVVVSATMNKDILEVTRKFMNDPVKILVKRDEISLEGIKQYVVNVDKEEWKFDTLCDIYDSLTITQCVIFCNTKKKVDWLSQRLIQSNFAVVSMHGDMKQEERDKVMNDFRTGHSRVLISTDVWARGIDVQQVSLVINYDLPEIIENYIHRIGRSGRFGRKGVAINFITKADLAKLREIEKFYSIKINPMPANFAELS

SEQ ID NO: 14 - protein, 604 aa, Saccharomyces cerevisiae
MAELSEQVQNLSINDNNENGYVPPHLRGKPRSARNNSSNYNNNNGGYNGGRGGGSFFSNNRRGGYGNGGFFGGNNGGSRSNGRSGGRWIDGKHVPAPRNEKAEIAIFGVPEDPNFQSSGINFDNYDDIPVDASGKDVPEPITEFTSPPLDGLLLENIKLARFTKPTPVQKYSVPIVANGRDLMACAQTGSGKTGGFLFPVLSESFKTGPSPQPESQGSFYQRKAYPTAVIMAPTRELATQIFDEAKKFTYRSWVKACVVYGGSPIGNQLREIERGCDLLVATPGRLNDLLERGKISLANVKYLVLDEADRMLDMGFEPQIRHIVEDCDMTPVGERQTLMFSATFPADIQHLARDFLSDYIFLSVGRVGSTSENITQKVLYVENQDKKSALLDLLSASTDGLTLIFVETKRMADQLTDFLIMQNFRATAIHGDRTQSERERALAAFRSGAATLLVATAVAARGLDIPNVTHVINYDLPSDVDDYVHRIGRTGRAGNTGLATAFFNSENSNIVKGLHEILTEANQEVPSFLKDAMMSAPGSRSNSRRGGFGRNNNRDYRKAGGASAGGWGSSRSRDNSFRGGSGWGSDSKSSGWGNSGGSNNSSWW

SEQ ID NO: 15 - protein, 406 aa, Homo sapiens
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI

SEQ ID NO: 16 - protein, 411 aa, Homo sapiens
MATTATMATSGSARKRLLKEEDMTKVEFETSEEVDVTPTFDTMGLREDLLRGIYAYGFEKPSAIQQRAIKQIIKGRDVIAQSQSGTGKTATFSISVLQCLDIQVRETQALILAPTRELAVQIQKGLLALGDYMNVQCHACIGGTNVGEDIRKLDYGQHVVAGTPGRVFDMIRRRSLRTRAIKMLVLDEADEMLNKGFKEQIYDVYRYLPPATQVVLISATLPHEILEMTNKFMTDPIRILVKRDELTLEGIKQFFVAVEREEWKFDTLCDLYDTLTITQAVIFCNTKRKVDWLTEKMREANFTVSSMHGDMPQKERESIMKEFRSGASRVLISTDVWARGLDVPQVSLIINYDLPNNRELYIHRIGRSGRYGRKGVAINFVKNDDIRILRDIEQYYSTQIDEMPMNVADLI

SEQ ID NO: 17 - protein, 195 aa, Leishmania infantum
MAQNDKIAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEI

SEQ ID NO: 18 - protein, 126 aa, Leishmania infantum
MAQNDKIAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSK

SEQ ID NO: 19 - protein, 237 aa, Leishmania infantum
MAQNDKIAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRDPVRILVKRESL

SEQ ID NO: 20 - protein, 213 aa, Leishmania infantum
IPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRDPVRILVKRESL

SEQ ID NO: 21 - protein, 378 aa, Leishmania infantum
PSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRDPVRILVKRESLTLEGIKQFFIAVEEEHKLDTLMDLYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGRKGVAINFVTEKDVELLHEIEAHYHTQIDELPVDFAAYLGE

SEQ ID NO: 22 - protein, 98 aa, Leishmania infantum
ETFVGGTRVQDDLRKLQAGVIVAVGTPGRVSDVIKRGALRTESLRVLVLDEADEMLSQGFADQIYEIFRFLPKDIQVALFSATMPEEVLELTKKFMRD

SEQ ID NO: 23 - protein, 133 aa, Leishmania infantum
ETFVGGTRVQDDLRKLQAGVIVAVGTPGRVSDVIKRGALRTESLRVLVLDEADEMLSQGFADQIYEIFRFLPKDIQVALFSATMPEEVLELTKKFMRDPVRILVKRESLTLEGIKQFFIAVEEEHKLDTLMDL

SEQ ID NO: 24 - protein, 208 aa, Leishmania infantum
FRFLPKDIQVALFSATMPEEVLELTKKFMRDPVRILVKRESLTLEGIKQFFIAVEEEHKLDTLMDLYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGRKGVAINFVTEKDVELLHEIEAHYHTQIDELPVDFAAYLGE

SEQ ID NO: 25 - protein, 167 aa, Leishmania infantum
LTLEGIKQFFIAVEEEHKLDTLMDLYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGRKGVAINFVTEKDVELLHEIEAHYHTQIDELPVDFAAYLGE

SEQ ID NO: 26 - protein, 143 aa, Leishmania infantum
LYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGRKGVAINFVTEKDVELLHEIEAHYHTQIDELPVDFAAYLGE

SEQ ID NO: 27 - protein, 403 aa, Artificial
  Protein LeIF of mutant Leishmania infantum having a substitution of the lysine in position 7 of motif I with alanine
MAQNDKIAPQDQDSFLDDQPGVRPIPSFDDMPLHQNLLRGIYSYGFEKPSSIQQRAIAPFTRGGDIIAQAQSGTGATGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRDPVRILVKRESLTLEGIKQFFIAVEEEHKLDTLMDLYETVSIAQSVIFANTRRKVDWIAEKLNQSNHTVSSMHAEMPKSDRERVMNTFRSGSSRVLVTTDLVARGIDVHHVNIVINFDLPTNKENYLHRIGRGGRYGRKGVAINFVTEKDVELLHEIEAHYHTQIDELPVDFAAYLGE

SEQ ID NO: 28 - protein, 9 aa, Artificial
  motif Q of eIF4A
GFEEPSAIQ

SEQ ID NO: 29 - protein, 8 aa, Artificial
  motif I of eIF4A
AQSGTGKT

SEQ ID NO: 30 - protein, 6 aa, Artificial
  motif Ia of eIF4A
PTRELA

SEQ ID NO: 31 - protein, 5 aa, Artificial
  motif Ib of eIF4A
TPGRV

SEQ ID NO: 32 - protein, 4 aa, Artificial
  motif II of eIF4A
DEAD

SEQ ID NO: 33 - protein, 8 aa, Artificial
  motif IV of eIF4A
VIFCNTRR

SEQ ID NO: 34 - protein, 9 aa, Artificial
  motif V of eIF4A
TDLLARGID

SEQ ID NO: 35 - protein, 8 aa, Artificial
  motif VI of eIF4A
HRIGRGGR

SEQ ID NO: 36 - protein, 8 aa, Artificial
  motif I of Prp2
GETGSGKT

SEQ ID NO: 37 - protein, 6 aa, Artificial
  motif Ia of Prp2
PRRVAA

SEQ ID NO: 38 - protein, 5 aa, Artificial
  motif Ib of Prp2
TDGML

SEQ ID NO: 39 - protein, 4 aa, Artificial
  motif II of Prp2
DEAH

SEQ ID NO: 40 - protein, 8 aa, Artificial
  motif IV of Prp2
LVFLTGQE

SEQ ID NO: 41 - protein, 9 aa, Artificial
  motif V Prp2
TNIAETSLT

SEQ ID NO: 42 - protein, 8 aa, Artificial
  motif VI of Prp2
QRAGRAGR

SEQ ID NO: 43 - protein, 8 aa, Artificial
  motif I of NS3
APTGSGKS

SEQ ID NO: 44 - protein, 5 aa, Artificial
  motif Ia of NS3
PSVAA

SEQ ID NO: 45 - protein, 5 aa, Artificial
  motif Ib of NS3
TYSTY

SEQ ID NO: 46 - protein, 4 aa, Artificial
  motif II of NS3
DECH

SEQ ID NO: 47 - protein, 8 aa, Artificial
  motif IV of NS3
LIFWHSKK

SEQ ID NO: 48 - protein, 9 aa, Artificial
  motif V of NS3
TDALMTGYD

SEQ ID NO: 49 - protein, 8 aa, Artificial
  motif VI of NS3
QRRGRTGR

SEQ ID NO: 50 - protein, 8 aa, Artificial
  motif I of SKi2
APTGAGKT

SEQ ID NO: 51 - protein, 6 aa, Artificial
  motif Ia of SKi2
PLKALS

SEQ ID NO: 52 - protein, 5 aa, Artificial
  motif Ib of SKi2
TTEVL

SEQ ID NO: 53 - protein, 4 aa, Artificial
  motif II of SKi2
DEVH

SEQ ID NO: 54 - protein, 8 aa, Artificial
  motif IV of SKi2
TFVFSRFG

SEQ ID NO: 55 - protein, 9 aa, Artificial
  motif V of SKi2
TETLALGIN

SEQ ID NO: 56 - protein, 8 aa, Artificial
  motif VI of SKi2
QLTGRAGR

SEQ ID NO: 57 - protein, 9 aa, Leishmania infantum
GFEKPSSIQ

SEQ ID NO: 58 - protein, 8 aa, Leishmania infantum
AQSGTGKT

SEQ ID NO: 59 - protein, 6 aa, Leishmania infantum
PTRELA

SEQ ID NO: 60 - protein, 5 aa, Leishmania infantum
TPGRV

SEQ ID NO: 61 - protein, 4 aa, Leishmania infantum
DEAD

SEQ ID NO: 62 - protein, 3 aa, Leishmania infantum
SAT

SEQ ID NO: 63 - protein, 9 aa, Leishmania infantum
KKFMRDPVR

SEQ ID NO: 64 - protein, 9 aa, Leishmania infantum
TDLVARGID

SEQ ID NO: 65 - protein, 8 aa, Leishmania infantum
HRIGRGGR

(19) United States
(12) Patent Application Publication
(10) Pub. No.: US 2011/0305667 A1
(43) Pub. Date: Dec. 15, 2011

(54) USE OF A DEAD-BOX RNA HELICASE FOR INDUCING CYTOKINE PRODUCTION

(75) Inventors: Ikram Guizani, Ariana (TN); Mourad Barhoumi, Tunis (TN); Amel Garnaoui, Tunis (TN); Nolen Kyle Tanner, Antony (FR)

(73) Assignees: INSTITUT PASTEUR DE TUNIS, Tunis Tunisia (FR); INSTITUT PASTEUR, Paris (FR)

(21) Appl. No.: 12/996,210

(22) PCT Filed: Jun. 5, 2009
(86) PCT No.: PCT/FR2009/000665

§ 371 Date: Mar. 14, 2011

(30) Foreign Application Priority Data
Jun. 6, 2008 (EP) 08290522.5

(51) Int. Cl. A61K 38/21; C12N 9/14; A61P 35/00; C07K 14/44; A61P 37/04; A61P 33/02; C12N 5/078; A61K 38/46

(52) U.S. Cl. 424/85.5; 435/375; 435/195; 424/274.1; 424/184.1; 530/324; 530/350

(57) ABSTRACT

The invention relates to the use of a DEAD-box RNA helicase from a yeast, a mammal, Leishmania infantum, or fragments thereof for inducing the production of cytokines by a peripheral blood mononuclear cell (PBMC) of a mammal, and to the applications thereof.

The present invention relates to the use of a DEAD-box RNA helicase from a yeast, from a mammal or from Leishmania infantum for inducing cytokine production by a peripheral blood mononuclear cell (PBMC) of a mammal and applications thereof.

The helicases are proteins capable of unwinding duplexes of DNA or of RNA by utilizing the energy released by the hydrolysis of nucleotide triphosphates (NTP) (Caruthers and McKay, 2002; Tanner and Linder, 2001). Two classes of helicases have been identified depending on the specificity of the substrate:
The DNA helicases, which fix DNA, unwind the complementary structures of DNA and dissociate DNA-protein interactions. They are ubiquitous proteins identified in various organisms ranging from viruses to higher eukaryotes. They are involved in many biological processes where the double-stranded structure of DNA must be unwound to the single-stranded form to serve as a substrate in various biological processes such as replication, recombination, transcription, and repair of DNA (Hall and Matson, 1999; Tuteja and Tuteja, 2004 a,b).
The RNA helicases, which unwind RNA duplexes and dissociate RNA-RNA and RNA-protein interactions. These proteins occur in all prokaryotic and eukaryotic organisms and in many viruses (Rocak and Linder, 2004). They are involved in all the processes of RNA metabolism: transcription, biogenesis of ribosomes, pre-mRNA splicing, nuclear export of mRNAs, initiation of translation and degradation of mRNAs (de la Cruz et al., 1999; Schmid and Linder, 1992; Silverman et al., 2003).

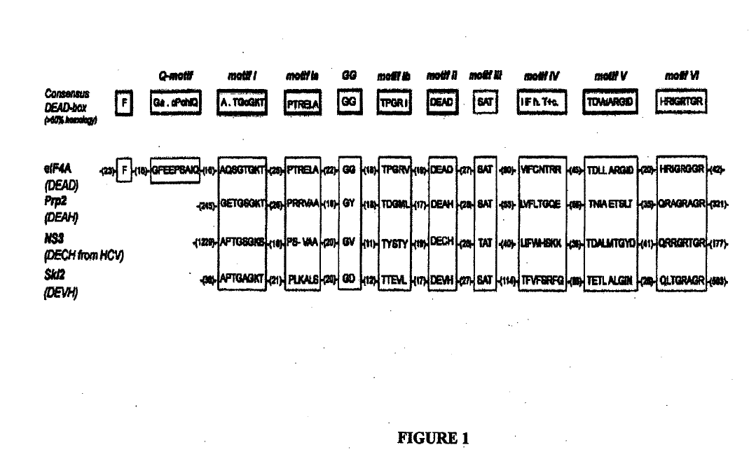

On the basis of comparisons of protein sequences, Gorbalenya and Koonin proposed a general classification of helicases in 5 main families (Gorbalenya and Koonin, 1993). Each family is characterized by a characteristic number and sequence of conserved motifs. All the families possess motifs for binding and hydrolysis of NTPs called walker A and B (Walker et al., 1982). The superfamilies 1 and 2 (SF1 and SF2) are the most represented. They include DNA and RNA helicases of bacteria, of archebacteria, of eubacteria, of eukaryotes and of viruses. They are characterized by 7 to 9 conserved motifs including 5 to 7 motifs in common (see FIG. 1). The superfamily SF3 includes small proteins (about 100 amino acids) of DNA and RNA viruses (Kadare and Haenni, 1997). They are characterized by 3 conserved motifs. The proteins of superfamily SF4 are DNA helicases that are involved in DNA replication in bacteria and bacteriophages (Ilyima et al., 1992). They are characterized by 5 conserved motifs H1, H1a, H2, H3 and H4. Patel and Picha showed that these proteins form hexamers that are capable of unwinding DNA in the 5′ to 3′ direction. The superfamily SF5 is represented by the transcription factor Rho (Patel and Picha, 2000). All these proteins have a similar structure, composed of a core containing motifs for fixation and hydrolysis of NTPs and nucleic acid binding motifs (Caruthers and McKay, 2002; Hall and Matson, 1999; Tanner and Linder, 2001; Ye et al., 2004).

The DExD/H-box proteins, belonging to the SF2 family, form the largest family of RNA helicases identified to date. They are present in all organisms, from bacteria to humans, as well as in certain viruses (de la Cruz et al., 1999; Rocak and Linder, 2004; Schmid and Linder, 1992). These proteins were identified for the first time when Linder et al. (1989) aligned the sequences of eight proteins that are homologs of the translation initiation factor of the eukaryotes eIF4A. The size of these proteins varies between 400 and 1200 residues. Subsequent studies have demonstrated the existence of nine conserved motifs: Q, I, Ia, Ib, II, III, IV, V and VI, which are contained in a central region, called the helicase core (Cordin et al., 2006; Tanner and Linder, 2001; see FIG. 1). The DEAD-box proteins take their name from the sequence of their motif II DEAD (for aspartate-glutamate-alanine-aspartate). The regions located in the N-terminal and C-terminal position of the helicase core are for their part very variable in size and in sequence.

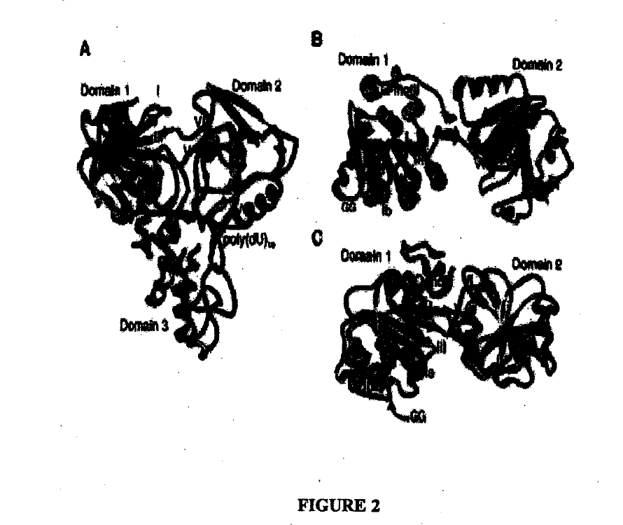

In the course of the last ten years, several structures of helicases of different superfamilies have been characterized. All the structures of the DEAD-box proteins as well as those of the helicases of the SF1 superfamily currently available, reveal a common theme, which consists of two globular domains, connected by a flexible linkage (see FIG. 2) (Story and Steitz, 1992). Domain 1 (D1) contains the ATP binding motifs (Q, I, II and III; see FIGS. 2B and C) whereas domain 2 (D2) contains the RNA binding motifs (IV, V and VI). As with the other helicases, all the conserved motifs in the DEAD-box proteins are localized at the β sheet-loop or α helix-loop transitions (Tanner and Linder, 2001). As well as the five β-sheets and five α-helices of the two domains 1 and 2, the DEAD-box proteins possess a β-sheet and two α-helices localized upstream of motif I which coil the Q motif (Tanner et al., 2003).

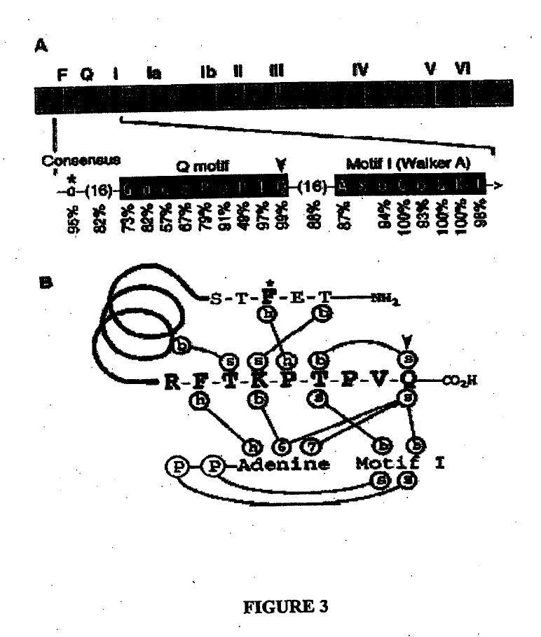

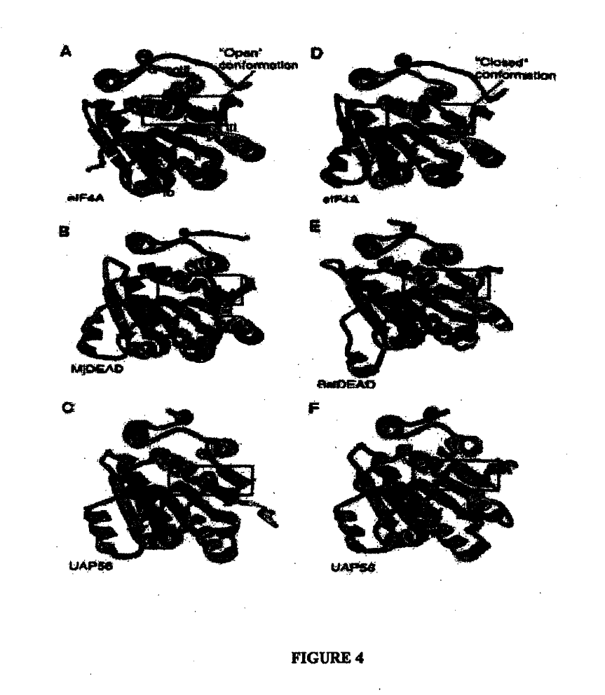

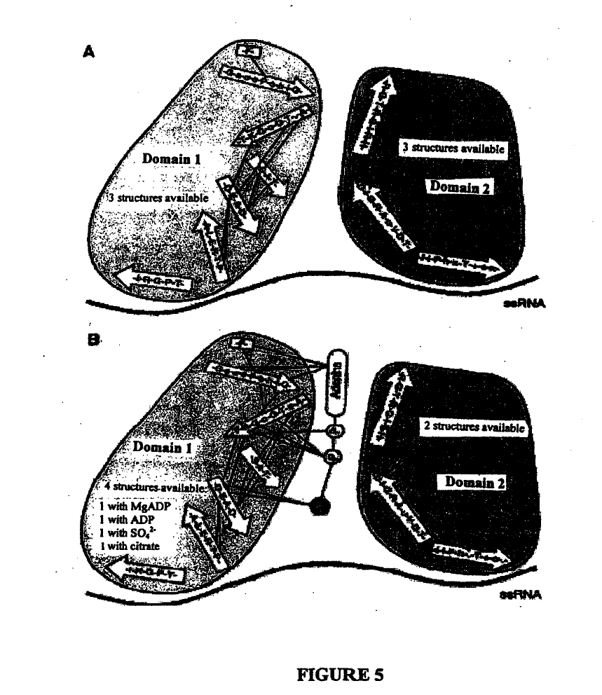

The DEAD-box proteins have, in their central part, nine conserved motifs, arranged identically and spaced with a similar number of amino acids. Mutation analyses combined with structure-function studies have made it possible to attribute roles to them.
Motif Q (SEQ ID NO: 1): discovered very recently, motif Q seems be a particular feature of the DEAD-box proteins (Tanner, 2003). This motif consists of nine amino acids which invariably include a glutamine (see FIGS. 1 and 3A), hence the name of the motif, and an isolated phenylalanine aromatic residue (F), highly conserved, located upstream, is added to these. The consensus sequence of motif Q is G-a-x-c-P-o-h-i-Q, where “a” represents F, W or Y, “x” represents any amino acid, “c” represents D, E, H, K or R, “o” represents S or T, “h” represents A, F, G, I, L, M, P, V, W or Y, and “i” represents I, L or V. The crystalline structures of proteins eIF4A and BstDEAD, obtained in the absence of a ligand, do not show interactions between motif Q and motif I (see FIGS. 4D and E), whereas the structures of protein eIF4A, in the presence of an ADP molecule, or the MjDEAD protein, in the presence of a sulfate ion, show several interactions between motif Q and motif I (see FIGS. 4A and B; Tanner et al., 2003; Cordin et al., 2004). In fact, glutamine and the alcohol residue (Thr) form a hydrogen bond with the first conserved glycine and the alcohol residue (Thr) of motif I. Moreover, motif Q can establish interactions with the bound nucleotide directly (see FIG. 2B). The conserved glutamine forms a hydrogen bond with positions N6 and N7 of the adenine base; moreover, the aromatic residue forms a hydrogen bond with the adenine base. Motif Q is thus a recognition motif of the adenine base, with the consequence that the DEAD-box proteins can only fix ATP (Tanner et al., 2003; Tanner, 2003). Motif Q is also necessary for the binding of single-stranded RNA as well as for the changes in conformation caused by the binding to and hydrolysis of ATP; accordingly, it has been proposed that motif Q functions as a regulator of ATPase activity by stimulating this activity only when the substrate is correctly bound at the protein level (Cordin et al., 2004).
Motif I (SEQ ID NO: 2): motif I or the motif Walker A exists in all the helicases as well as in several NTPases or ATPases (Gorbalenya et al., 1989; Walker et al., 1982). Its consensus sequence is A-x-o-G-o-G-K-T, where “x” represents any amino acid, and “o” represents independently S or T (see FIG. 1). This motif is involved in the binding of NTP by formation of a hydrogen bond with the β and γ phosphates of the nucleotide and interacts with the Mg²⁺ ion (Caruthers and McKay, 2002). The crystalline structures of DEAD-box proteins obtained in the absence of a ligand show that the conserved lysine interacts with the first aspartate and the glutamate of motif II (Johnson and McKay, 1999; Carmel and Matthews, 2004), whereas in the presence of ATP it only interacts with the glutamate (Benz et al., 1999; Story et al., 2001). Comparison of the crystalline structures of eIF4A and MjDEAD obtained in the presence of an ADP molecule or of a sulfate ion bound at the level of the binding site of the nucleotide respectively, with the crystalline structures of eIF4A, BstDEAD or UAP56 obtained in the absence of a ligand, shows a particular architecture of motif I. In the presence of a ligand, motif I adopts a conformation called “open” whereas in the absence of a ligand, it adopts a conformation called “closed”, resulting in steric inhibition of binding to ATP (Benz et al., 1999; Story et al., 2001; Zhao et al., 2004) (see FIG. 4). This change in conformation at the level of motif I seems to be characteristic of the DEAD-box helicases. In fact the crystalline structures of the proteins of the SF1 superfamily: PCRA, UVRB and Rep (Korolev et al., 1997; Subramanya et al., 1996; Theis et al., 1999; Velankar et al., 1999) or that of the helicase part of the NS3 protein of hepatitis C (Kim et al., 1998; Yao et al., 1997) obtained in the presence or in the absence of a ligand show that motif I always adopts the “open” conformation. Studies by mutagenesis have shown that this motif is important for the ATPase and ATP binding activities. Mutations of the alanine in first position, of the lysine and of the threonine cancel the ATPase activity (Barhoumi et al., 2006; Cordin et al., 2004; Pause and Sonenberg, 1992; Rocak et al., 2005; Rozen et al., 1990; Tanner et al., 2003).
The motifs Ia (SEQ ID NO: 3), Ib (SEQ ID NO:4) and the GG doublet: these motifs form part of domain 1 but they do not participate directly in binding and in hydrolysis of ATP. Biochemical studies of the protein eIF4A (Rogers et al., 2002) as well as of the crystalline structure of the helicase part of the NS3 protein of hepatitis C obtained in the presence of a poly dU (see FIG. 2A; Kim et al., 1998) show that these motifs are necessary for binding to RNA. Substitution of the alanine of motif Ia in certain DEAD-box proteins cancels the ATPase and helicase activities (Svitkin et al., 2001). In contrast, the same mutations in DEAH-box proteins such as Prp22 do not affect the growth of yeast strains (Shneider et al., 2004). In 1989, Linder et al. showed that the two glycines form part of the conserved elements that define the family of the DEAD-box proteins (Linder et al., 1989). From a structural standpoint, these two glycines are localized at the loop positioned between motifs Ia and Ib (see FIGS. 2B and C), thus facilitating the formation of a “sharp turn” in this loop. It has been suggested that they participate in protein-protein interactions such as binding between eIF4A and eIF4G (Benz et al., 1999). Mutations of these two glycines are lethal for yeast (Schmid and Linder, 1992).
Motif II DEAD (SEQ ID NO: 5): this motif represents a version of the Walker B motif of the ATP binding proteins (Walker et al., 1982). The consensus sequence of the motif for the SF1 and SF2 superfamilies is DExx. In the DEAD-box proteins, the sequence of motif II (DEAD) is dominant for the entire family (see FIG. 1; Linder et al., 1989). The DE residues are conserved in all the DEAD-box proteins, and mutations in one of these two residues reduce or inactivate the ATPase and helicase activities without altering the binding to RNA (lost et al., 1999; Pause and Sonenberg, 1992). The crystalline structures of the DEAD-box proteins show that the Walker B motif interacts with the phosphates of the NTP in the β and γ position via an Mg²⁺ ion. Story et al. proposed that the binding of ATP closes the crack between the two domains 1 and 2, thus bringing the last aspartate of motif II (from 13 Å to 5 Å) closer to the conserved histidine of motif VI. The same aspartate forms a hydrogen bond with the amino acids serine and threonine of motif III (see FIG. 5; Benz et al., 1999; Caruthers et al., 2000; Shi et al., 2004; Story et al., 2001).
Motif III SAT (SEQ ID NO: 6): mutations in the motif SAT (Ser-Ala-Thr) of protein eIF4A and certain DEAH-box homologous proteins cancel the RNA helicase activity without altering the fixation to and hydrolysis of ATP as well as binding to RNA (Pause and Sonenberg, 1992; Schwer and Mezaros, 2000). Recently, it was shown that substitutions of the serine and the threonine with an alanine in the DEAD-box protein Has1 of yeast partially dissociate the helicase and ATPase activities (Rocak et al., 2005). In the crystalline structure of the helicase part of the NS3 protein of hepatitis C (Kim et al., 1998, see FIG. 2A), motif III appears as a part of the flexible linkage between domains 1 and 2, whereas in the crystalline structures of the DEAD-box proteins: MjDEAD (Story et al., 2001), BstDEAD (Carmel and Matthews, 2004) and UAP 56 (Zhao et al., 2004), it belongs to domain 1 and it is separated from domain 2 by a helix-loop-β-sheet structure. As already pointed out, motif III interacts with the motif DEAD in the presence or in the absence of the nucleotide. In contrast to what is observed for the PcrA protein of the SF1 superfamily (Subramanya et al., 1996; Velanker et al., 1999), motif III of the DEAD-box proteins does not interact with the bound ATP. Korolev et al. propose that motif III of the proteins of the SF1 superfamily serves as a relay which transmits the effects of fixation to and hydrolysis of ATP to the RNA binding motifs IV and V (Korolev et al., 1997). Probably, motif III exerts the same function in the DEAD-box proteins.
Motif IV (SEQ ID NO: 7): the crystalline structures established on this motif show that this motif is localized in the C-terminal part of domain 2 in a region where the NS3 protein binds to the oligo-dU. All the structural data obtained for the helicases of the SF2 superfamily suggest a common mode of binding to the nucleic acid via the phosphodiester backbone and the capacity of the last two arginines of motif IV of protein eIF4A to bind to the single-stranded RNA (Caruthers and McKay, 2002). The first residues of motif V are very close to the hydrophobic residue of motif IV (see FIG. 5; Caruthers et al., 2000; Shi et al., 2004; Story et al., 2001), which suggests communication between motifs IV and V. Recently, Banroques et al. showed, by studies of mutagenesis, that the conserved phenylalanine residue of motif IV is necessary for cooperation between fixation to RNA and hydrolysis of ATP (Banroques et al., 2008).
Motif V (SEQ ID NO: 8): motif V, together with motifs Ia, Ib and IV, is involved in binding to RNA. It is localized at the loop connecting domains 1 and 2 in the RNA binding region (see FIGS. 1 and 4). Its consensus sequence is T(D/N)xxARGiD, where “x” represents independently any amino acid, and “i” represents I, L or V (see FIG. 1). The crystalline structure of eIF4A (see FIG. 2B; Caruthers et al., 2000) shows that the conserved arginine of motif V interacts with motif II or with the bound nucleotide. Moreover, the last aspartate interacts directly with the ribose of ATP (Caruthers et al., 2000; Caruthers and McKay, 2002). The crystalline structures established for the DEAD-box proteins, in the presence or in the absence of the ligand, show different interactions (see FIG. 5). In fact, in the crystalline structures established in the presence of the ligand for the proteins MjDEAD and UAP56, the last aspartate interacts with the first or second arginine, or the threonine of motif VI (Johnson and McKay, 1999; Shi et al., 2004; Story et al., 2001; Zhao et al., 2004). In the crystalline structure established for the C-terminal fragment of protein eIF4A, the first aspartate interacts with the first arginine of motif VI (Johnson and McKay, 1999). However, in that of the protein UAP56 crystallized in the presence of ADP, the last aspartate of motif V interacts with the first arginine of motif VI (see FIG. 5; Shi et al., 2004). Comparison of the structure of motif V in the DEAD-box proteins with that of the helicase part of the NS3 protein of hepatitis C shows that the two motifs adopt the same folding. Consequently, as for protein NS3, motif V of the DEAD-box proteins would be involved in regulation of the hydrolysis of ATP by transmission of the signal of binding to RNA to the ATPase domain. Mutations in motif V of the DEAD-box protein Prp28 greatly inhibit growth of the yeast strains, suggesting that the two conserved arginines and the aspartate play an important role in the activity in vivo of protein prp28 (Chang et al., 1997).
Motif VI (SEQ ID NO: 9): motif VI (HRiGRzGR; where “i” represents independently I, L or V and where “z” represents T, G or S) of the DEAD-box proteins is localized at the interface between the two domains 1 and 2 (see FIGS. 2 and 5); it is important for the ATPase and RNA-binding activities. Mutations at the level of the conserved residues arginine or histidine greatly reduce RNA binding and ATPase activity and consequently cancel the helicase activity (Pause et al., 1993; Rogers et al., 2002). The crystalline structure of eIF4A shows that the histidine residue interacts with the second aspartate of motif II. The same interactions are observed between the homologous glutamine of motif V and the histidine of motif II of the DExH-box, UvrB and NS3 proteins (Yao et al., 1997; Kim et al., 1998). Caruthers et al. suggest that the second and third arginine can bind to the phosphate-γ of ATP (Caruthers et al., 2000).

The DEAD-box proteins are, like the other helicases, capable of fixing and hydrolyzing a nucleotide triphosphate. In the case of the DEAD-box proteins, only ATP can be fixed and hydrolyzed because of the specific interactions between the Q motif and the adenine base (Tanner et al., 2003; Tanner, 2003). The various biochemical studies conducted on these proteins have shown that their affinity for ATP is moderate, with a Km generally between 80 and 1000 μM (Lorsh and Herschlag, 1998a). Moreover, their kinetic constants are variable from 3-6 min⁻¹ (eIF4A: Lorsh and Herschlag, 1998a; Has1: Rocak et al., 2005) to 600 min⁻¹ (Ded1: Iost et al., 1999; DbpA: Kossen and Uhlenbeck, 1999). The significance of these differences is unclear and it is possible that absence of post-translational modifications, absence of a partner or absence of specific substrates accounts for them. The majority of DEAD-box proteins only hydrolyze ATP in the presence of RNA. This activity is sometimes influenced by the nature (sequence and/or length) of the RNA substrate. In fact, stimulation of the ATPase activity of the DEAD-box proteins of E. coli SrmB, RhlE and CsdA depends on the length of the RNA used as substrate (Bizebard et al., 2004). Most of the DEAD-box proteins do not display specificity of the RNA substrate, in vitro. In fact, specificity for a given RNA seems rather to be afforded by motifs or domains located outside of the helicase core. In the case of the DEAD-box protein of E. coli DbpA, its ATPase activity is stimulated by RNA 23S, more precisely by a fragment of 153 nucleotides containing helix 92 of domain V of this RNA (Fuller-Pace et al., 1993; Tsu and Uhlenbeck, 1998; Tsu et al., 2001). The protein DbpA would interact specifically with helix 92 via its C-terminal domain and nonspecifically with the adjacent region via its helicase core (Tsu et al., 2001; Kossen et al., 2002). The DEAD-box proteins are regarded as RNA helicases, although this activity has only been demonstrated for some of them. Like the DNA helicases, the DEAD-box proteins are capable of unwinding duplexes constituted of at least one strand of RNA either from 5′ to 3′ or from 3′ to 5′, when they are tested in vitro. Most of these DEAD-box proteins require the presence of single-stranded RNA regions either at 5′ or at 3′ of the paired region, probably to be charged there (Rocak and Linder, 2004). The protein eIF4A is capable of unwinding short RNA/RNA or RNA/DNA duplexes; in contrast, it is incapable of unwinding DNA/DNA duplexes. Moreover, it is also capable of unwinding flush-ended RNA duplexes, suggesting that it can interact directly with double-stranded RNA (Rogers et al., 2001a). This property is also observed for the DEAD-box protein of E. coli RhlE (Bizebard et al., 2004). Certain DEAD-box proteins, such as eIF4A, p68 and RhlE are said to be bidirectional, because, in vitro, they are capable of unwinding indiscriminately duplexes bearing a single-stranded end at 3′ or at 5′ (Bizebard et al., 2004; Huang and Liu, 2002; Rogers et al., 2001a). The DEAD-box proteins, like the majority of helicases, are not very processive. Only proteins p68 (162 bp duplex, Hirling et al., 1989), p72 (36 to 46 bp, Rossler et al., 2001) and RhlE (Bizebard et al., 2004) display moderately processive activity. The helicase activity depends on the stability of the duplex used. The stability of the duplexes frequently used is ΔG° between −15 and −30 kcal/mol and the length varies between 10 and 25 bp. Rogers et al., using duplexes of variable length and stability, showed that the protein eIF4A is capable of unwinding several base pairs (Rogers et al., 2001a). Recently, Bizebard et al. showed that the length of the single-stranded region, the minimum size of which varies depending on the proteins, is important for helicase activity (Bizebard et al., 2004).

Certain helicase proteins display, in vitro, pairing activity of homologous sequences. Thus, as well as their helicase activity, the two DEAD-box proteins p68 and p72 are capable, in vitro, of catalyzing the pairing of complementary strands, when they are present in excess relative to the substrate (Rossler et al., 2001). By combining these two activities, they can catalyze, in vitro, rearrangements of secondary structures of RNA, which are moreover too stable to be eliminated by their helicase activity alone (Rossler et al., 2001). Flores-Rozas and Hurwitz showed that in the presence of nonhydrolyzable analogs of ATP, the DEAD-box protein II/Gu catalyzes an intramolecular pairing reaction. The concentration of ATP plays a crucial role in this reaction. At low concentration, the RNA helicase II/Gu promotes intramolecular duplex formation, whereas at high concentrations of ATP, the enzyme acts like RNA helicase and eliminates the secondary structures (Flores-Rozas and Hurwitz, 1993). This pairing activity resides outside of the helicase core in the C-terminal domain, rich in Gly-Arg (Valdez et al., 1997). This might also be the case with the DEAD-box proteins p68 and p72, which possess N-terminal and C-terminal domains rich in glycine and arginine (Lamm et al., 1996). The pairing and RNA-helicase activities are separated physically and functionally. Mutations in motifs II and III of the DEAD-box protein II/Gu cancel the RNA-helicase activity without altering the pairing activity. Moreover, deletion of the pairing domain does not inhibit the helicase activity of the enzyme (Valdez et al., 1997; Valdez, 2000). Protein eIF4B, which stimulates the helicase and ATPase activities of protein eIF4A (Rogers et al., 2001b), also possesses pairing activity (Altmann et al., 1995), proving once again that the two activities are physically separate.

Various models have been proposed for the helicase activity (Tanner and Linder, 2001; Caruthers and McKay, 2002; Singleton and Wigley, 2002). The two models most discussed are: “inchworm” and “active rolling”.

The DEAD-box proteins are also involved in all processes of RNA metabolism (transcription, maturation, transport, translation, biogenesis of ribosomes, RNA interference, stability and degradation of RNAs).

In the course of transcription, several helicases become associated with the transcripts. However, they only seem to play an active role during the late stages of splicing or transport (Linder and Stutz, 2002; Silverman et al., 2003). Recent studies have shown that certain DEAD-box proteins participate in the regulation of transcription by interacting with other transcription factors (Gillian and Svaren, 2004; Yan et al., 2003). However, the usefulness of ATPase and helicase activities in the transcription process is not clearly established. However, Wilson et al. showed that the helicase cores of the DEAD-box proteins p68 and p72 suppress transcription whereas their C-terminal region stimulates transcription (Wilson et al., 2004). Recently, Bates et al. (2005) showed that the DEAD-box protein p68 activates the transcription of p53 following a DNA aberration (Bates et al., 2005).

Splicing is a process that takes place in several stages involving two reactions of transesterification and structural rearrangements in the stage of assembly of the spliceosome which requires the energy derived from hydrolysis of NTP. The precise role of RNA helicases in assembly of the spliceosome is not known, but it is generally assumed that they are involved in unwinding the small duplexes formed between snRNA and pre-mRNA, which is the case with the DExH-box protein, Prp 22. A mutant in motif III of this protein has a phenotype that is lethal at temperatures below 30° C. and a slowing of growth at 34° C. and 37° C. In vitro, the mutant protein is capable of hydrolyzing ATP, but is incapable of unwinding RNA duplexes and liberating mRNA from the spliceosome, suggesting that the loss of helicase activity is the cause of the lethal phenotype. A second mutation in motif Ib, which restores the helicase activity and the dissociation of mRNA from the spliceosome, behaves as an intragenic suppressor, suggesting that helicase activity is necessary in this stage (Schwer and Meszaros, 2000). The majority of DEAD-box proteins that are implicated in the process of mRNA splicing are involved in the early stages of assembly of the spliceosome, which is the case for the DEAD-box protein Prp5. Recently, it was reported that this protein is capable of binding to snRNA U1 and U2 (Xu et al., 2004). The DEAD-box protein Prp5 uses energy derived from the hydrolysis of ATP to rearrange the local RNA-RNA or RNA-protein interactions, which permit the snRNA U2 complex to rejoin the snRNA U1 complex. The DEAD-box protein Prp28 uses the energy released by hydrolysis of ATP to destabilize the snRNA U1 complex and replace it with the snRNA U6 complex at the splicing site (Staley and Gutherie, 1999). The DEAD-box protein p68 is involved in dissociation of the snRNA U1 complex from site 5′, necessary for splicing according to an ATP dependent mechanism.

A large proportion of DEAD-box RNA helicases participate in the biogenesis of ribosomes, in which their helicase or RNPase activity might provide fine regulation of the organization of the multiple RNA-RNA or RNA-protein interactions transiently established in the course of biogenesis and maturation of ribosomal RNAs (Luking et al., 1998; Kressler et al., 1999; Rocak and Linder, 2004). Only the DEAD-box protein Has1 is involved simultaneously in the biogenesis of the 40S and 60S subunits (Emry et al., 2004). Mutations in motif I of the protein Has1, as well as its depletion, affect the biosynthesis of rRNA 18S of the 40S subunits, by slowing or inhibiting cleavage at sites A0, A1 and A2.

The transport of mRNA from the nucleus to the cytoplasm through the nuclear pores involves specific proteins which bind to the nascent mRNA. The RNA helicases that are involved in this process could remodel the mRNP complexes before or during passage through the nuclear pores (Silverman et al., 2003). They could also be involved in dissociation of the nuclear factors of the mRNA to permit export of the latter, or preparation of the messenger in the first cycle of translation (Rocak and Linder, 2004). The DEAD-box protein Dbp5 participates in this process. Double hybrid and co-immunoprecipitation experiments have identified several partners, including Gle1, a factor for exporting the RNA associated with the nuclear pore, and GFd1/ymr255, a factor that interacts both with Dbp5 and GLe1 (Tseng et al., 1998; Hodge et al., 1999; Schmitt et al., 1999). The yeast and human Dbp5 proteins also interact with Nup159, a compound of the nuclear pore complex (Zhao et al., 2002). In yeast, the ATPase activity of the DEAD-box protein Sub2 is necessary for its detachment from the mRNA and its replacement with the mRNA transport factor Mex 67 (Strasser and Hurt, 2001).

Nonsense mediated decay (NMD) is a surveillance mechanism that breaks down mRNAs containing a premature termination codon (PTC). Several proteins which form part of the exon junction complex are involved in this mechanism (Reed and Hurt, 2002). Recently, Ferraiuolo et al. (2004) showed that the DEAD-box protein eIF4AIII belongs to the exon junction complex (EJC) and that it binds to RNA during splicing (Chan et al., 2004; Ferraiuolo et al., 2004; Shibuya et al., 2004). The protein eIF4AIII interacts with eIF4G and eIF4B but, in contrast to eIF4AI and eIF4AII, it inhibits translation (Li et al., 1999). Its role in the nucleus has not yet been established but certain authors suggest a role in RNA transport (Palacios et al., 2004) and in nonsense mediated decay (Ferraiuolo et al., 2004; Palacios et al., 2004; Shibuya et al., 2004).

The DEAD-box protein Ded1 is also involved in initiation of translation, probably independently of eIF4A (Chuang et al., 1997; de la Cruz et al., 1997; Iost et al., 1999; Linder, 2003). The precise role of Ded1 in the process of initiation of translation is not yet known, but it might be involved in the process of displacement of the small subunit 40S up to the initiation codon for eliminating secondary structures on the mRNA or random pairings between mRNA and tRNA, or imperfect codon/anticodon interactions (Linder, 2003). A mutation in the helicase core of the protein Ded1 selectively inhibits the translation of polymerase 2A, thus suppressing replication of the RNA2 genome of the brome mosaic virus whereas it assures general translation of the cell. This suggests that the protein Ded1 might have regulatory functions separate from its general role in translation or that the mutant protein has a helicase activity that is weak, but nevertheless sufficient to assure cellular translation but not translation of RNA polymerase 2A (Noueiry et al., 2000).

The use of immunotherapeutic approaches aiming to improve the immune responses of the host to developing tumors has been demonstrated. They include immunomodulating cytokines such as TNF-alpha, the INFs of type I and of type II, IL-2, IL-12, IL-15 and IL-18, which are among the most potent inducers of antitumor activity according to preclinical studies. IL-12 in particular is receiving particular attention because of its central role in the regulation of innate and adaptive immune responses. By itself, it can induce powerful anticancer effects, but can also act in synergy with other cytokines to increase its immunoregulatory and antitumor activities (Weiss et al., 2007). Among the approaches currently being considered, we may mention the use of recombinant IL-12 in experimental models or in clinical trials (Shiratori et al., 2007; Lenzi et al., 2007); the transduction of dendritic cells by a recombinant adenovirus expressing IL-12 and their use as vaccine (He et al., 2008); or even the use of microspheres loaded with DNA coding for IL-12 (Son and Kim, 2007) or with synergistic combinations of cytokines IL-12 and TNF-alpha (Sabel et al., 2007).

The Leishmania are flagellated protozoa, belonging to the order Kinetoplastidae and to the family Trypanosomatidae. They are responsible for leishmaniases.

The protein LeIF was identified by screening a genomic DNA database of promastigotes of Leishmania braziliensis with serum from patients with mucocutaneous leishmaniasis due to L. braziliensis (Skeiky et al., 1995). Sequence comparison with those present in the databases enabled it to be identified as being homologous to the translation initiation factor eIF4A (Skeiky et al., 1995). It is a cytoplasmic protein of 403 amino acids, with molecular weight of 45.3 kDa, whose transcripts are detected both at the promastigote stage and in the amastigote (Skeiky et al., 1998; Salay et al., 2007) and whose gene is present in duplicate in the Leishmania genome (Myler et al., 1999). Proteomic studies have shown that the protein LeIF is overexpressed in the amastigote and promastigote stage (Bente et al., 2003; Nugent et al., 2004); it is highly conserved between the different species of the genus Leishmania (Barhoumi et al., 2006; Skeiky et al., 1998). It has the motifs characteristic of the DEAD-box proteins (Barhoumi et al., 2006). However, in experiments on yeast, it was shown that the protein LeIF of Leishmania does not make up for loss of the protein eIF4A in yeast but interacts with the translation initiation factors (eIF4G) unproductively, generating a negative dominant phenotype (Barhoumi et al., 2006).

The protein LeIF of the species L. braziliensis has the capacity to induce the production of IFN-γ and of TNF-α by PBMCs (peripheral blood mononuclear cells) of patients with cutaneous leishmaniasis, mucosal leishmaniasis or diffuse cutaneous leishmaniasis, and of IL-12 both by PBMCs of patients and of uninfected individuals (Skeiky et al., 1995). This protein is also capable of inducing the secretion of cytokines IL-12, IL-10 and TNF-α by the antigen-presenting cells: macrophages and dendritic cells obtained from normal individuals (Probst et al., 1997). The cytokine inducing activity of the protein LeIF seems to be localized in the N-terminal region (1-226) (Probst et al., 1997). These results were confirmed by the same authors in the murine model. Thus, Skeiky et al. (1998) showed that the recombinant protein LeIF of the species Leishmania major is capable of stimulating the secretion of IFN-γ by the splenic cells of BALB/c mice infected with L. major, of promoting the development of TH1 clones in BALB/c mice immunized with this protein in the absence of adjuvant and finally of stimulating the production of IFN-γ by splenocytes of SCID mice, by an IL-12-dependent mechanism. They also showed that this activity was maintained with the N-terminal part of the protein (1-226) and lost with the C-terminal part (196-403). The protein LeIF is one of the constituents of a second-generation vaccine against Leishmania composed of three fused proteins of Leishmania, which was described as being immunogenic and conferring a significant degree of protection in the mouse immunized with Leishmania major or L. infantum (Coler et al., 2002; 2007), but which does not seem to be protective in mice immunized with L. braziliensis (Salay et al., 2007). This vaccine is still undergoing clinical trials in humans. The protein LeIF has also been used in immunotherapy in a patient with mucocutaneous leishmaniasis (Badaro et al., 2001). It is also capable of increasing the expression of molecules B7-1 and CD54 (ICAM-1) (molecules of co-stimulation involved in interaction between T cells and antigen presenting cells) on the surface of macrophages and of dendritic cells from healthy donors, tested in vitro (Probst et al., 1997).

The present invention set itself the goal of providing novel molecules capable of modulating the production of cytokines by the host cells of mammals, for treating or preventing infectious diseases or notably cancers.

Thus, the inventors used monocytes derived from PBMCs of healthy donors primed or not with IFN-γ for testing the capacity of recombinant proteins to induce the production of the cytokines IL-12 (in particular IL-12p70), IL-10 and TNF-α, so as to demonstrate their immunomodulating properties.

Five DEAD-box recombinant proteins possessing ATPase and RNA helicase biochemical activities, present in yeast or in mammals, were tested for their immunomodulating properties:
hueIF4A: translation initiation factor in mammals, it is assumed to unwind the secondary structures at the 5′UTR end of mRNAs to permit the recruitment and optionally the displacement of the small subunit 40S of the ribosome (Rogers et al., 2002; Sonenberg and Diver, 2003). It has 406 amino acids and a molecular weight of 46.1 kDa. It has 56.1% identity with the protein LeIF of L. infantum;
eIF4AIII: mammalian protein of 406 amino acids and with molecular weight of 46.8 kDa. It has 55.6% identity with the protein LeIF of L. infantum. It is involved in the processes of splicing and degradation of mRNA (nonsense mediated mRNA decay) (Chan et al., 2004; Ferraiulo et al., 2004; Shibuya et al., 2004; Palacios et al., 2004);
yeIF4A: translation initiation factor in yeast (Linder, 2003). It has 394 amino acids and a molecular weight of 44.5 kDa. It has 54.6% identity with the protein LeIF of L. infantum;
FAL1: nuclear protein of yeast. It has 399 amino acids and a molecular weight of 45.2 kDa. It has 52.6% identity with the protein LeIF of L. infantum. It is involved in the biogenesis of ribosomes (Kressler et al., 1997); and
Ded1: yeast protein of 603 amino acids organized in three parts: the helicase core, an N-terminal extension of 144 amino acids and a C-terminal extension of 110 amino acids. It has 31% identity with the protein LeIF of L. infantum. It is involved in initiation of translation in yeast (Chuang et al., 1997; Kressler et al., 1997; lost et al., 1999; Linder, 2003).

Surprisingly, the inventors showed that the DEAD-box proteins and notably hueIF4A, eIF4AIII, yeIF4A, FAL1 and Ded1, even though they each display less than 57% homology with the protein LeIF of L. infantum, also possess immunomodulating activity and are capable of inducing the secretion of the cytokines IL-12, IL-10 and TNF-alpha.

The inventors also tested fragments of the protein LeIF of L. infantum, 26-403, 1-226, 1-237, 1-195, 25-237, 129-226, 129-261, 196-403 and 237-403, as well as the mutant protein LeIFK76A having a substitution of the lysine in position 76 (in the consensus motif I of the protein LeIF of L. infantum) with an alanine, for their immunomodulating properties.

The inventors showed that all of the fragments of LeIF of L. infantum tested induce the production of IL-12p70 by monocytes from healthy individuals in vitro, and that the mutation, in motif I, of the lysine in position 76 to alanine, which cancels the ATPase activity of the protein LeIF, does not affect its activity for inducing the cytokines IL-12p70, IL-10 and TNF-α.

The present invention therefore relates to the use of a yeast or mammalian DEAD-box RNA helicase comprising in its central part, arranged from (1) to (9) from the N-terminal end to the C-terminal end, the nine motifs of amino acids defined by the following consensus motifs: (1) motif Q (SEQ ID NO: 1), (2) motif I (SEQ ID NO: 2), (3) (SEQ ID NO: 3), (4) motif Ib (SEQ ID NO: 4), (5) motif II (SEQ ID NO: 5), (6) motif III, (SEQ ID NO: 6), (7) motif IV (SEQ ID NO: 7), (8) motif V (SEQ ID NO: 8) and (9) motif VI (SEQ ID NO: 9), of wild-type or having a mutation in consensus motif I (SEQ ID NO: 2), or of a fragment of the latter comprising either the six consensus motifs Q (SEQ ID NO: 1), I (SEQ ID NO: 2), Ia (SEQ ID NO: 3), Ib (SEQ ID NO: 4), II (SEQ ID NO: 5) and III (SEQ ID NO: 6) but not the three consensus motifs IV (SEQ ID NO: 7), V (SEQ ID NO: 8) and VI (SEQ ID NO: 9), or the three consensus motifs IV, V and VI (defined previously) but not the six consensus motifs Q, I, Ia, Ib, II and III defined previously, for inducing the production in vitro or ex vivo of at least one cytokine by a peripheral blood mononuclear cell (PBMC), preferably a monocyte, of a mammal.

“DEAD-box RNA helicase” means a protein possessing RNA helicase activity and comprising in its central part, arranged from the N-terminal end to the C-terminal end from (1) to (9), nine motifs of amino acids defined by the following consensus motifs:

(1) motif Q, of amino acid sequence G-a-x-c-P-o-h-i-Q (SEQ ID NO: 1), where “a” represents F, W or Y, “x” represents any amino acid, “c” represents D, E, H, K or R, “o” represents S or T, “h” represents A, F, G, I, L, M, P, V, W or Y, and “i” represents I, L or V;

(2) motif I, of amino acid sequence A-x-o-G-o-G-K-T (SEQ ID NO: 2), where “x” represents any amino acid, and “o” represents S or T;

(3) motif Ia, of amino acid sequence P-T-R-E-L-A (SEQ ID NO: 3);

(4) motif Ib, of amino acid sequence T-P-G-R-i (SEQ ID NO: 4), where “i” represents I, L or V;

(5) motif II, of amino acid sequence D-E-A-D (SEQ ID NO: 5);

(6) motif III, of amino acid sequence S-A-T (SEQ ID NO: 6);

(7) motif IV, of amino acid sequence i-i-F-C/h-x-T-x-b-c (SEQ ID NO: 7), where “i” represents independently I, L or V, “h” represents A, F, G, I, L, M, P, V, W or Y, “x” represents independently any amino acid, “b” represents H, K or R, and “c” represents D, E, H, K or R;

(8) motif V, of amino acid sequence T-D/N-x-x-A-R-G-i-D (SEQ ID NO: 8), where “x” represents independently any amino acid, and “i” represents I, L or V; and

(9) motif VI, of amino acid sequence H-R-i-G-R-z-G-R (SEQ ID NO: 9), where “i” represents independently I, L or V and where “z” represents T, G or S,

said motifs being spaced apart by a number of amino acids defined according to the motifs in question.

The RNA helicase activity of a protein can be determined by any method known by a person skilled in the art. As nonlimiting examples, it is possible to use the methods described by Rogers et al., 1999 and Cordin et al., 2004.

According to an advantageous embodiment, said RNA helicase possesses ATPase activity. The ATPase activity of a protein can be determined by any method known by a person skilled in the art. As nonlimiting examples, it is possible to use the methods described by Cordin et al., 2004; Rocak et al., 2005 or Tanner et al., 2003.

The DEAD-box RNA helicase can be:
a protein isolated from a yeast or from a mammal,
a recombinant protein, or
a synthetic protein.

According to another advantageous embodiment, said RNA helicase has a mutation at the lysine in position 7 of the above motif I. Preferably, the mutation is a substitution of said lysine with any other amino acid, preferably with alanine.

According to a preferred embodiment of the present invention, said yeast belongs to the genus Saccharomyces, and preferably to the species Saccharomyces cerevisiae.

According to another preferred embodiment of the present invention, said mammalian DEAD-box RNA helicase is a human DEAD-box RNA helicase.

Advantageously, the DEAD-box RNA helicase of yeast is selected from the proteins yeIF4A (SEQ ID NO: 12), FAL1 (SEQ ID NO: 13) and Ded1 (SEQ ID NO: 14), and the mammalian DEAD-box RNA helicase is selected from the proteins hueIF4A (SEQ ID NO: 15) and eIF4AIII (SEQ ID NO: 16).

Other yeast or human DEAD-box proteins can also be employed; examples of such proteins are mentioned above. We may notably mention the proteins P68 (mammalian DEAD-box protein; Iggo R D and Lane D P, EMBO J., 1989) and Has1 (yeast DEAD-box protein; Rocack et al., N.A.R., 2005).

According to another advantageous embodiment of the present invention, said cytokine is selected from the group comprising IL-12 (in particular IL-12p70), TNF-alpha and IL-10.

According to a particular embodiment of the present invention, said peripheral blood mononuclear cell is activated beforehand by interferon gamma (IFN-γ) to induce production of IL-12.

As a nonlimiting example, IL-12p70 can be produced by monocytes pre-activated with IFN-γ, preferably for 12 hours, then stimulated with the yeast or mammalian DEAD-box RNA helicase as defined above, preferably for 24 hours.

The present invention also relates to the use of a yeast or mammalian DEAD-box RNA helicase of the wild type or having a mutation in consensus motif I or of a fragment of the latter, as defined above, for preparing a medicinal product intended for treating or preventing infectious diseases or cancers.

Said medicinal product is notably useful as a vaccination adjuvant for inducing an immune response of the Th1 type.

Said immune response of the Th1 type preferably consists of production of at least one of the cytokines IL-12 and TNF-alpha.

More particularly, the infectious diseases are parasitic infections, preferably infections induced by an intracellular parasite and more preferably leishmaniases.

The present invention also relates to a pharmaceutical composition comprising a yeast or mammalian DEAD-box RNA helicase of the wild type or having a mutation in consensus motif I, or of a fragment of the latter, as defined above, and at least one pharmaceutically acceptable vehicle.

The pharmaceutically acceptable vehicles are those used conventionally and are selected depending on the method of administration used for the treatment envisaged.

The present invention also relates to products containing (i) a yeast or mammalian DEAD-box RNA helicase of the wild type or having a mutation in consensus motif I, or of a fragment of the latter, as defined above and (ii) IFN-γ, for simultaneous, separate or sequential use in the treatment or prevention of cancer and of infectious diseases, preferably parasitic infections, and more preferably leishmaniases.

The present invention also relates to a fragment of the protein LeIF of Leishmania infantum of sequence SEQ ID NO: 11, selected from:
fragments of the latter comprising either the six motifs Q (SEQ ID NO: 57), I (SEQ ID NO: 58), Ia (SEQ ID NO: 59), Ib (SEQ ID NO: 60), II (SEQ ID NO: 61) and III (SEQ ID NO: 62) but not the three consensus motifs IV (SEQ ID NO: 63), V (SEQ ID NO: 64) and VI (SEQ ID NO: 65), or the three consensus motifs IV, V and VI (defined previously) but not the six consensus motifs Q, I, Ia, Ib, II and III defined previously, and
fragments of the latter corresponding to the amino acids 1-195 (SEQ ID NO: 17), 1-237 (SEQ ID NO: 19), 25-237 (SEQ ID NO: 20), 26-403 (SEQ ID NO: 21), 129-226 (SEQ ID NO: 22), 237-403 (SEQ ID NO: 25) or 261-403 (SEQ ID NO: 26) of said protein LeIF of L. infantum.

The present invention also relates to a fragment of the protein LeIF of Leishmania infantum as defined above, preferably the fragments of sequence SEQ ID NO: 17, 19, 20, 21 and 26, or of the protein LeIF of Leishmania infantum of the wild type (SEQ ID NO: 11) or mutated in the consensus motif I for use as a medicinal product.

According to another advantageous embodiment, the protein LeIF of L. infantum has a mutation at the lysine in position 7 of the above motif I, corresponding to the lysine residue in position 76 of the protein. Preferably, the mutation is a substitution of said lysine with any other amino acid, preferably with alanine.

The protein LeIF of mutant Leishmania infantum having a substitution of the lysine in position 7 of motif I defined above with alanine is designated LeIFK76A. Its amino acid sequence is shown in the sequence SEQ ID NO: 27.

According to another advantageous embodiment of this use, the medicinal product is intended for treating or preventing infectious diseases or cancers.

The medicinal product can moreover be used as a vaccination adjuvant for inducing an immune response of the Th1 type, which preferably consists of production of at least one of the cytokines IL-12 (in particular IL-12p70) and TNF-alpha.

The present invention also relates to products containing (i) a fragment of the protein LeIF of Leishmania infantum as defined above, preferably the fragments of sequence SEQ ID NO: 17, 19, 20, 21 and 26, or the protein LeIF of Leishmania infantum as defined above and (ii) IFN-γ, for simultaneous, separate or sequential use in the treatment or prevention of cancer and of infectious diseases, preferably parasitic infections, more preferably infections induced by an intracellular parasite and more preferably leishmaniases.

The present invention also relates to a fragment of the protein LeIF of Leishmania infantum as defined above, preferably the fragments of sequence SEQ ID NO: 17, 19, 20, 21 and 26, or the protein LeIF of Leishmania infantum as defined above for inducing the production in vitro or ex vivo of IL-12 by a peripheral blood mononuclear cell (PBMC), preferably a monocyte, of a mammal, preferably a human.

The present invention also relates to a method of modulating the production of cytokines, characterized in that it comprises stimulation of the production of cytokines IL-12, TNF-α and IL-10, by cells of a host and notably of monocytes, said method comprising the simultaneous, separate or sequential administration of the DEAD-box proteins or their fragments, as defined above, and of IFN-γ.

In addition to the foregoing embodiments, the invention further comprises other embodiments, which will become clear from the following description, which refers to examples illustrating the immunomodulating properties of yeast or mammalian DEAD-box RNA helicases, or of fragments of the protein LeIF of L. infantum, as well as the appended drawings, in which:

FIG. 1 illustrates the conserved motifs of the DEXD/H-box RNA helicase proteins. This figure shows the sequences of the conserved motifs of the proteins: eIF4A of yeast (DEAD-box protein), Prp22 (DEAH-box protein), NS3 (hepatitis C virus helicase of the DECH family) and SKi2 (DExH) as well as the spacings that separate the motifs. Various symbols are used for representing the consensus sequence of the motifs of the DEAD-box proteins: − o: S, T; − I: I, L, V; − a: F, w, Y; − c: D, E, H, K, R; −h: A, F, G, I, L, M, P, V, W, Y; +: H, K, R; −u: A, G; − .: any residue.

FIG. 2 illustrates the crystalline structures of helicases of superfamily 2 (SF2) represented by the proteins NS3, eIF4A and MjDEAD. (A) The crystalline structure of the protein NS3 is obtained in the presence of a bound oligonucleotide (Kim et al., 1998, accession number PDB 1A1V). The co-crystallized sulfate ion is not shown. (B) the crystalline structure of the whole protein eIF4A (Caruthers et al., 2000, accession number PDB 1FUU). (C) the structure of the protein MjDEAD (Story et al., 2001, accession number PDB 1HV8).

FIG. 3 illustrates the Q motif of the DEAD-box proteins. (A) Conservation of the Q motif of the DEAD-box proteins (according to Tanner et al., 2003). (B) Schematic representation of the interactions within the Q motif and between motif Q, motif I and bound ADP. The sequence is that of the protein Ded1 (Cordin et al., 2004).

FIG. 4 illustrates the two open and closed conformations of motif I of the DEAD-box proteins. (A) structure of the N-terminal part of the protein eIF4A obtained in the presence of bound ADP (Benz et al., 1999; accession number PDB: 1QDE); the bound ADP is not shown. (B) structure of domain 1 of the protein MjDEAD (Story et al., 2001; accession number PDB: 1HV8). (C) the structure of domain 1 of the protein UAP56 crystallized in the presence of ADP (Shi et al., 2004; accession number PDB: 1XTJ). (D) the structure of domain 1 of the protein eIF4A crystallized in the absence of the nucleotide (Caruthers et al., 2000; accession number PDB: 1FUU). (E) the structure of domain 1 of the protein BstDEAD crystallized in the absence of the nucleotide (Carmel and Matthews, 2004; accession number PDB: 1QOU). (F) the structure of domain 1 of the protein UAP56 crystallized in the absence of ADP (Shi et al., 2004; accession number PDB: 1XTI). (A) to (C) the rectangle shows the open conformation of motif I. (D) to (F) the rectangle shows the closed conformation of motif I.

FIG. 5 illustrates the intramolecular interactions of the DEAD-box proteins in the presence and in the absence of the nucleotide and shows schematically the interactions between the conserved motifs in domains 1 and 2 based on the structural data of the proteins eIF4A, MjDEAD, BstDEAD and UAP56. The arrows indicate the orientation of the motifs. (A) interactions in the absence of the nucleotide (Caruthers et al., 2000; Carmel and Matthews, 2004; Shi et al., 2004; Zhao et al., 2004). (B) interactions between the motifs in the presence of the ligand (ADP, sulfate or citrate ion; Benz et al., 1999; Story et al., 2001; Shi et al., 2004).

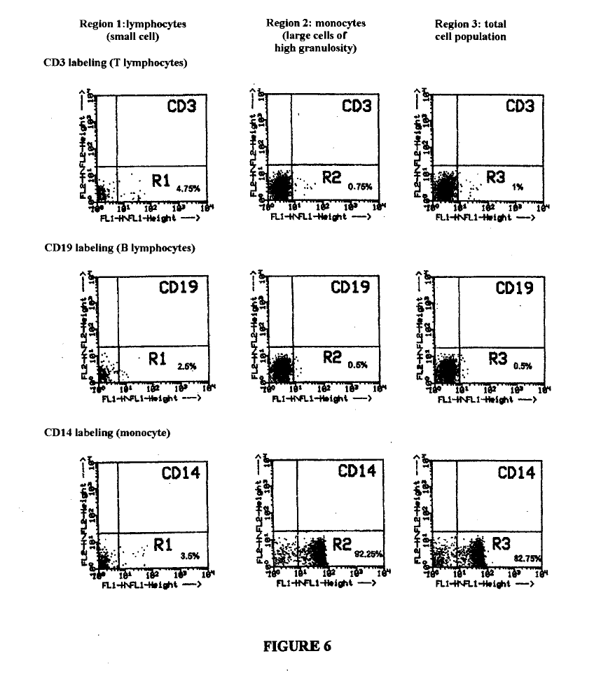

FIG. 6 illustrates analysis of the purity of the monocyte population by flow cytometry. The quantity of fluorescence emitted by the cells is shown in the diagrams, and is determined relative to a positivity threshold fixed by means of isotypical monitoring. The T and B lymphocytes (CD3⁺ and CD19⁺ respectively) do not exceed 2% of the cellular population in R3, the monocytes are present to more than 80% in this region, and represent 92.25% of the cells of region R2.

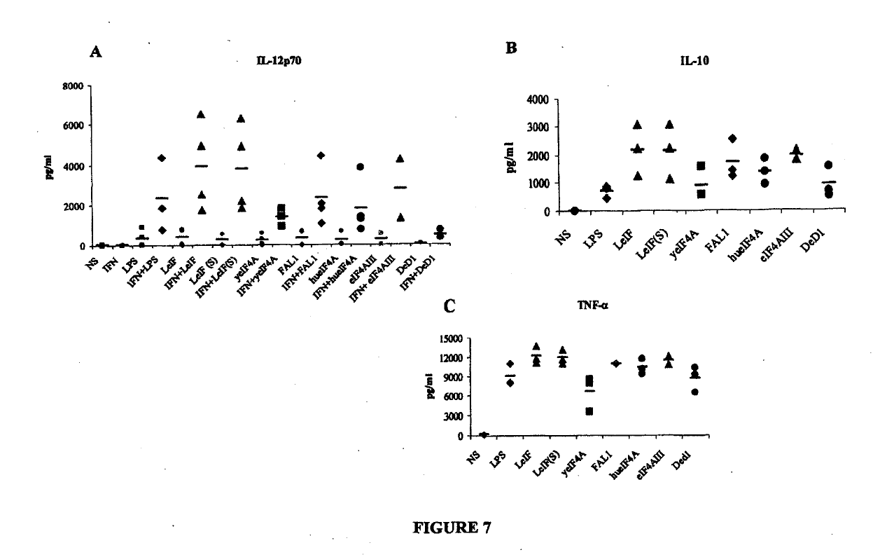

FIG. 7 illustrates induction, by the proteins yeIF4A, FAL1 and Ded1 of yeast and human hueIF4A and eIF4AIII, homologs of the protein LeIF, of the secretion of cytokines IL-12p70 (A), IL-10 (B) and TNF-α (C) by human monocytes in vitro. NS: monocytes that are not stimulated. IFN: monocytes primed by IFN-γ at a final concentration of 3000 U/ml for 12 h, LPS: monocytes stimulated for 18 h with LPS at 1 μg/ml.

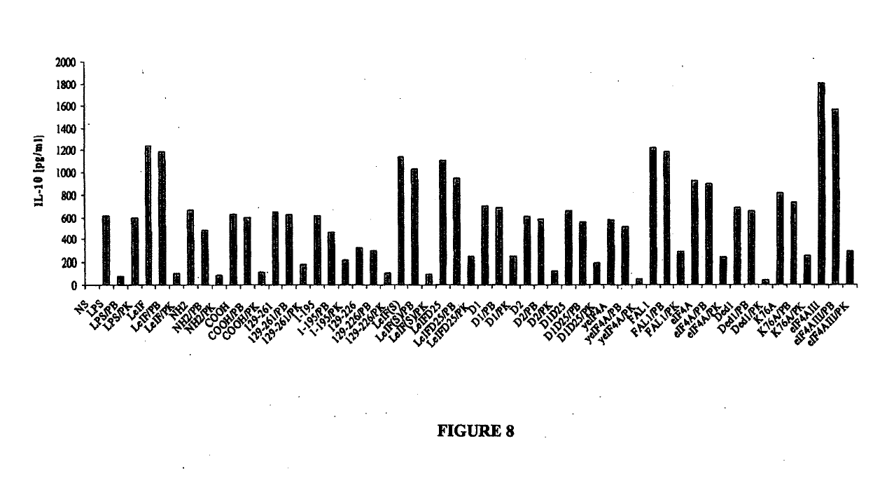

FIG. 8 illustrates the activity of induction of the production of cytokines by different recombinant proteins in the presence of proteinase K and of polymixin B. The monocytes purified from PBMCs from one and the same healthy individual were stimulated by different recombinant proteins alone, or co-incubated with polymixin B (10 μg/ml), or pretreated for 30 min at 42° C. with proteinase K (100 μg/ml). As controls, the monocytes were stimulated with LPS (1 μg/ml) alone or in the presence of polymixin B (10 μg/ml), or after pretreatment for 30 min at 42° C. with proteinase K (100 μg/ml). The culture supernatants were collected after incubation for 18 h and the levels of IL-10 were determined by sandwich ELISA. NS: monocytes that were not stimulated.

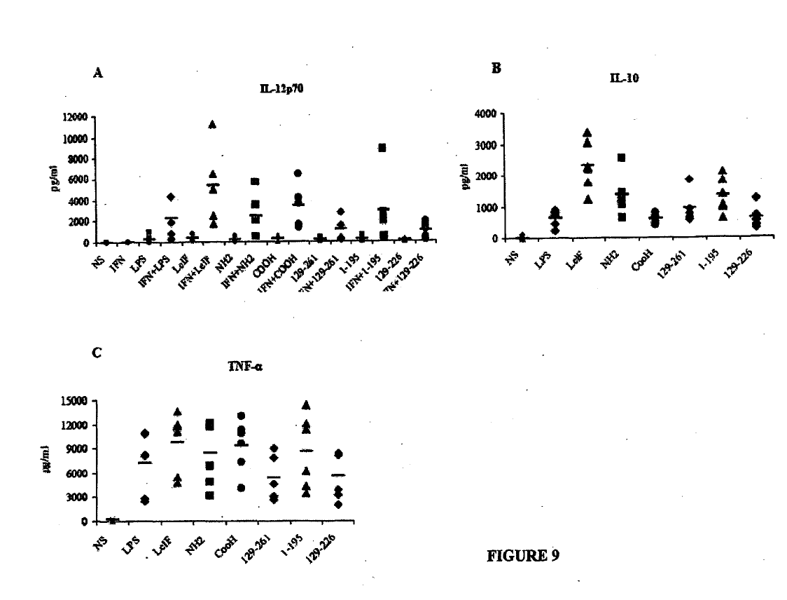

FIG. 9 illustrates the induction, by different fragments of the protein LeIF of L. infantum, of the production of cytokines IL-12p70 (A), IL-10 (B) and TNF-α (C) by human monocytes in vitro. NS: monocytes that were not stimulated, IFN-γ: monocytes stimulated with IFN-γ at a final concentration of 3000 U/ml. LPS: monocytes stimulated with LPS at 1 μg/ml, for 18 h.

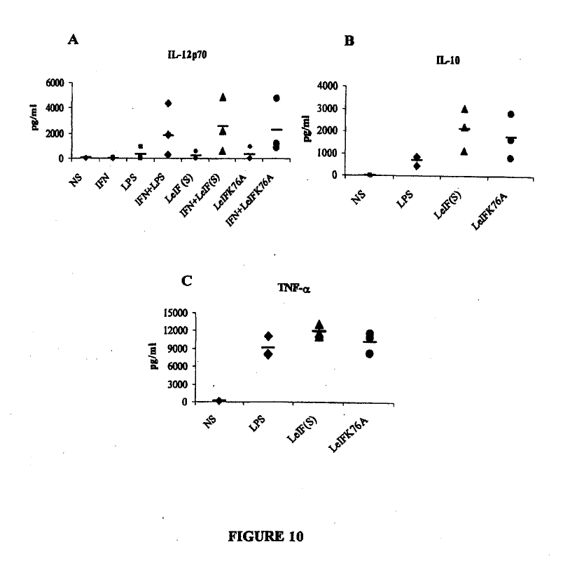

FIG. 10 illustrates the induction, by the protein LeIFK76A (SEQ ID NO: 27), of the production of cytokines IL-12p70 (A), IL-10 (B) and TNF-α (C) by human monocytes in vitro. NS: monocytes that were not stimulated, IFN: monocytes primed with IFN-γ (3000 U/ml) for 12 h, LPS: monocytes stimulated for 18 h with LPS at 1 μg/ml

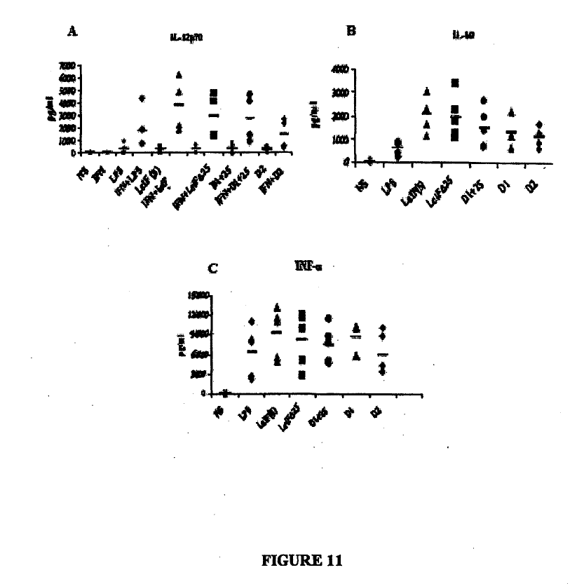

FIG. 11 illustrates the induction, by different domains of the protein LeIF of L. infantum, of the production of cytokines IL-12p70 (A), IL-10 (B) and TNF-α (C) by human monocytes in vitro. NS: monocytes that were not stimulated, IFN-γ: monocytes stimulated with IFN-γ at a final concentration of 3000 U/ml for 12 h, LPS: monocytes stimulated for 18 h with LPS at 1 μg/ml;

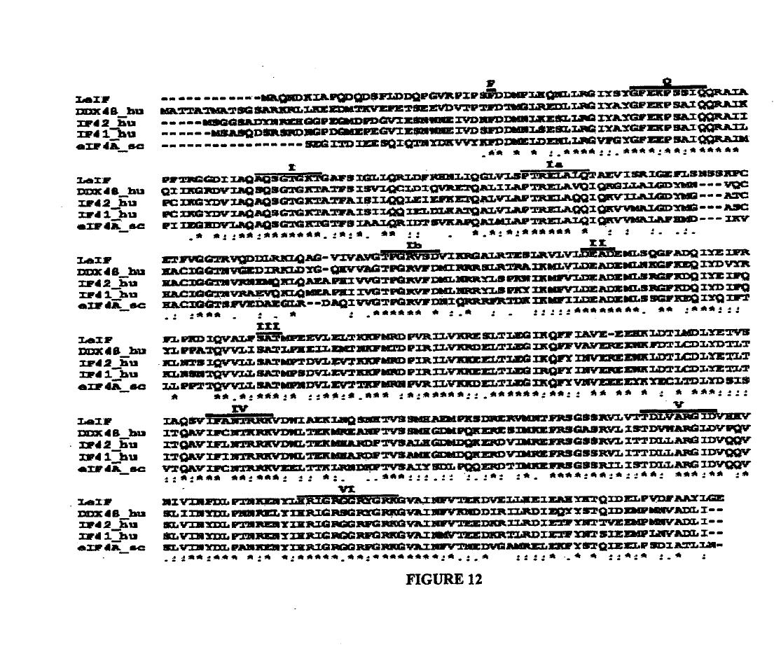

FIG. 12 illustrates comparison of the sequence of the protein LeIF of L. infantum with the sequences of the following human (Hu) or yeast (Sc) proteins: DDX48_hu, IF42_hu, IF41_hu and eIF4A_sc (as described in Barhoumi et al., 2006).

### EXAMPLE 1

### Evaluation of the Effect of Various Yeast or Mammalian DEAD-Box RNA Helicases as Inducers of the Production of Monocyte Cytokines

I. Material

I.1. Donors

Human monocytes were isolated from blood donations (Tunis Blood Transfusion Center), obtained on anticoagulant (citrate-phosphate-dextrose CPD) from healthy adult volunteers.

I.2. Recombinant Proteins
Yeast DEAD-box RNA helicases: yeIF4A (SEQ ID NO: 12), FAL1 (SEQ ID NO: 13) and Ded1 (SEQ ID NO: 14) (amino acid sequences available respectively under accession numbers P10081, Q12099 and P06634 in the UniProtKB/Swiss-Prot database). The nucleotide sequences encoding these proteins are available respectively under accession numbers gi|83722562, gi|93117368 and gi|84626310 in the GENBANK database.
Mammalian DEAD-box RNA helicases: hueIF4A (SEQ ID NO: 15) and eIF4AIII (SEQ ID NO: 16) amino acid sequences available respectively under accession numbers P60842 and P38919 in the UniProtKB/Swiss-Prot database). The nucleotide sequences encoding these proteins are available respectively under accession numbers gi|4503528 and gi|41327777 in the GENBANK database.
LeiF of L. infantum, Aymen isolate (SEQ ID NO: 11) (Barhoumi et al., 2006). The nucleotide sequence encoding this protein is shown in sequence SEQ ID NO: 10.

II. Methods

II.1.1 Isolation of Peripheral Blood Mononuclear Cells (PBMC)

The blood, obtained from healthy donors, on CPD, is centrifuged at 1800 rev/min for 20 min. After removal of the plasma, the blood is diluted in RPMI 1640 medium supplemented with 100 U/ml of penicillin, 100 μg/ml of streptomycin and 2 mM of L-glutamine (RPMI/PS/GLU). The plasma is decomplemented at 56° C. for 30 min, then clarified by centrifugation at 3000 rev/min for 10 min at room temperature. The mononuclear cells (PBMCs) are isolated on a density gradient constituted of Ficoll hypaque (Amersham). The blood is deposited on the Ficoll, then centrifuged at 1600 rev/min for 20 min at room temperature. The red blood cells and the polynuclear cells pass through the Ficoll and are found in the pellet, whereas the PBMCs remain at the interface of the Ficoll. The ring of PBMC is taken, diluted in RPMI/PS/Glu and washed three times by centrifugation at 1600 rev/min for 10 min at room temperature. The cells are counted on a Malassez plate.

II.1.2. Purification of the Monocytes from the PBMCs

Sterile culture flasks are treated with a 2% gelatin solution by incubation for 2 h at 37° C., then the excess gelatin is aspirated and the flasks are placed in a stove at 56° C. overnight. These flasks can be stored at room temperature for some days until they are used.

The decomplemented autologous plasma (AP) is deposited in gelatinized culture flasks. This plasma is rich in fibronectin, a protein that attaches to the gelatin and permits adherence of the monocytes via the fibronectin receptor CD49 expressed on the surface of these cells. After removing the plasma from this suspension, the PBMCs at 2.10⁶/ml are resuspended in RPMI/PS/Glu supplemented with 5% autologous plasma, and are then put back in the flasks. These cultures are incubated for 1 hour at 37° C. in the presence of 5% CO₂. The nonadherent cells are removed by washing gently three times, and the adherent cells are detached after incubation for 15 min at 37° C. with a buffer containing 50% of a solution of PBS 10 mM EDTA and 50% of RPMI/10% FCS or AP. The detached cells are washed to remove all traces of EDTA. A fraction of the cells purified in this way is taken for determining the purity of the monocyte preparation, by flow cytometry.

II.1.3. Determination of the Purity of the Monocytes by FACS

10⁶ cells are recovered from the monocyte preparation by adherence, washed and resuspended in PBS 1% BSA, 0.01% NaN₃ then distributed at a rate of 2.10⁵ cells/tube in hemolysis tubes. Antibodies (Becton Dickinson) labeled with fluorescein (FITC) or with phycoerythrin (PE) and specific for the surface markers CD14 (present on the majority of monocytes), CD19 (membrane marker of B lymphocytes), and CD3 (present on T lymphocytes) are added at a rate of 5 μl/tube and incubated for 30 min at 4° C., protected from the light. The nonspecific fluorescence of each antibody, which can be effected through the Fc of the IgGs, is monitored by incubation of the cells with an antibody of the same isotype as the specific antibody, used for identifying the various cellular populations, and labeled with the same fluorochrome as this antibody. The cellular suspensions are then washed twice, the pellets are resuspended in a fixing solution (PBS-paraformaldehyde (PFA) 0.3%) and then analyzed by FACS.

II.2. Induction of Secretion of Cytokines and Assay by Sandwich ELISA

II.2.1. Stimulation in Vitro

The human monocytes are resuspended at a rate of 10⁶ cells/ml in RPMI/PS/Glu 3% AP and distributed in 24-well culture plates (Nunc), at a rate of 5×10⁵ monocytes/well (10⁶ cells/ml). The cells in culture are stimulated either with LPS (1 μg/ml) or with the various recombinant proteins (10 μg/ml), for assay of IL-10 and of TNF-α. After incubation at 37° C. for 18 hours in the presence of 5% CO₂, the cells are centrifuged at 3000 rpm for 10 min and the culture supernatants are recovered and stored at −80° C. until they are used.

For assay of IL-12, the monocytes are primed with IFN-γ (3000 U/ml) for 12 hours before adding LPS (1 μg/ml) or the various recombinant proteins (10 μg/ml). After incubation for 24 h, the supernatants are collected and stored at −80° C. until they are used.

To verify absence of contaminants of bacterial origin (LPS) in the preparations of recombinant proteins, control cultures were added in the same experimental conditions. For this, LPS (1 μg/ml) and the various recombinant proteins (10 μg/ml) are incubated in the presence of proteinase K, at a final concentration of 100 μg/ml, at 42° C. for 30 min. Afterwards, the enzyme is inactivated at 75° C. for 10 min, then these preparations are used for stimulating the monocytes. Polymixin B, which has the effect of blocking the action of LPS, is added directly to the cells, to a final concentration of 10 μg/ml.

II.2.2. Assay of Cytokines by ELISA (Enzyme Linked Immuno Sorbent Assay)

The levels of cytokines secreted (IL-10, TNF-α and IL-12) in the culture supernatants are quantified by ELISA of the sandwich type. A first antibody or capture antibody, adsorbed on the plastic of a microtitration plate (Maxisorp Nunc), binds specifically to the cytokine to be assayed. A second antibody or detection antibody coupled to an enzyme becomes fixed to the capture antibody/cytokine complexes. This fixation is detected by an enzymatic reaction, which transforms a chromogenic substrate to a colored product. The levels of cytokines secreted in the culture supernatants are determined from a standard range recorded with known concentrations of a standard cytokine.

The ELISA test is performed according to the following stages:
The wells of a microtitration plate are sensitized with 100 μl/well of purified anti-human cytokine monoclonal antibodies (Becton Dickinson) diluted to 1/250 in bicarbonate buffer pH 9.6 (0.1 M NaHCO₃ and 0.1 M Na₂CO₃), overnight at +4° C.
The antibodies that have not fixed are removed by washing three times with PBS, 0.05% Tween 20 pH 7.0 (PBS-T).
The sites that remained free are saturated with 200 μl/well of PBS-skim milk 5% for 1 hour at room temperature. The plate is washed twice with PBS-T.
The culture supernatants and the recombinant cytokine, used at the dilutions recommended by the supplier (Becton Dickinson) and constituting the standard range (7.8, 15.6, 31.3, 62.5, 125, 250, 500 and 1000 pg/ml), are deposited at a rate of 100 μl/well and incubated for two hours at room temperature. The plate is washed 5 times with PBS-T.
100 μl of a detecting solution is deposited per well. This solution contains the biotinylated anti-human cytokine monoclonal antibody and the enzyme (Avidin-Horseradish peroxidase conjugate (Avidin-HRP)), diluted in buffer PBS-skim milk (dilution to 1/500, for the anti-IL-10 antibody, and to 1/250 for the anti-IL-12p70 and anti-TNF-α antibodies and to 1/250 for the Avidin-HRP enzyme). After incubation for one hour at room temperature, the excess of biotinylated antibodies is removed by washing 7 times with PBS-T.
The peroxidase activity is detected by adding 100 μl/well of the solution containing the substrate of the enzyme: TMB prepared extemporaneously. It contains two reagents in equal volume: reagent A: contains hydrogen peroxide and reagent B: contains 3,3′,5,5′-tetramethylbenzidine (TMB). The substrate solution turns pale blue after mixing the two reagents and turns blue in the presence of peroxidase. The plate is incubated for 30 min at room temperature in darkness.
The reactions are stopped by adding 50 μl/well of 2N H₂SO₄. After incubation for 30 min at room temperature, the blue color changes to yellow and the optical densities are measured with a spectrophotometer at a wavelength of 450 nm.

II.2.3. Statistical Analyses

The results were analyzed using the statistical analysis software S plus (version 6.2) by applying the paired t-test. This test compares the difference of the means with zero. It is calculated according to the following equation

$$t = \frac{\overline{d}}{\frac{s}{\sqrt{n}}} ,$$

where

d: the mean of the difference

S²: the variance of the samples

n: sample size

t: Student t.

III. Results

III.1. Evaluation of the Purity of the Monocytes

Human monocytes were isolated from the PBMCs of healthy individuals by adherence on a gelatin support treated with autologous plasma. A fraction (10⁶) of the cells recovered was analyzed by flow cytometry, to estimate their purity and their viability. For this, the cells were incubated with antibodies coupled to a fluorochrome and that recognize the surface antigens: CD14 (marker of monocytes), CD3 (marker of T cells) or CD19 (marker of B cells). The amount of specific fluorescence emitted by the labeled cells is correlated with the expression of these markers.

Immunofluorescent labeling made it possible to estimate the percentage of the monocytes and that of the contaminating, T and B, lymphocytes. FIG. 6 shows that the total cellular population represented in region R3 is constituted to 82.7% of monocytes (CD14⁺ cells) and only 2% of T lymphocytes (CD3⁺ cells) and B lymphocytes (CD19⁺ cells).

III.2. The Yeast Proteins yeIF4A, FAL1 and Ded1 and the Mammalian Proteins hueIF4A and eIF4AIII Induce the Secretion of Cytokines IL-12p70, IL-10 and TNF-α by Human Monocytes in Vitro.

The monocytes purified from the PBMCs from 5 healthy donors were activated, or not, by IFN-γ then stimulated by the recombinant proteins LeIF (control), yeIF4A, FAL1, hueIF4A, eIF4AIII and Ded1 to final concentrations of 10 μg/ml.

The results presented in FIG. 7A show that the recombinant proteins yeIF4A, FAL1, hueIF4A, eIF4AIII and Ded1 induce the production of levels of IL-12p70 significantly higher than those induced in the culture supernatants of the monocytes that were not stimulated (34.17±17.77 pg/ml) or stimulated with IFN-γ alone (35.53±17.84 pg/ml) or with the recombinant proteins alone (p<0.05, cf. Table 1 below).

The proteins yeIF4A, FAL1, hueIF4A and eIF4AIII induce levels of IL-12p70 (1391.3±266.98; 2338.37±738.34; 1820.28±681.35 and 2790±1467.5 pg/ml, respectively) comparable to those induced by the soluble or insoluble protein LeIF (3805.9±1069.1 and 3921.6±1094 pg/ml; p>0.05, cf. Table 1). However, the protein Ded1 induces the secretion of levels of IL-12p70 (497.6±79.19 pg/ml) that are significantly lower than those induced by soluble or insoluble protein LeIF (p<0.05, Table 1).

Table 1 below shows statistical analysis of the differences observed for the levels of production of IL-12p70 in the culture supernatants of the monocytes stimulated with the yeast proteins yeIF4A, FAL1 and Ded1 and mammalian eIF4AI and eIF4AIII. The p values determined by the paired t test are shown.

TABLE 1
IL-12p70

| | | | IFN-γ + | IFN-γ + | IFN-γ + | IFN-γ + | IFN-γ + | IFN-γ + | IFN-γ + |
|---|---|---|---|---|---|---|---|---|---|
| | NS | IFN-γ | LPS | LeIF | LeIF (S) | yeIF4A | FAL1 | hueIF4A | eIF4AIII |
| IFN-γ | 0.3132 | | | | | | | | |
| IFN-γ + LPS | 0.0108 | 0.0108 | | | | | | | |
| IFN-γ + LeIF | 0.0189 | 0.0186 | 0.3049 | | | | | | |
| IFN-γ + LeIF (S) | 0.0193 | 0.0190 | 0.3373 | 0.2936 | | | | | |
| IFN-γ + yeIF4A | 0.0436 | 0.0435 | 0.1611 | 0.1343 | 0.1222 | | | | |
| IFN-γ + FAL1 | 0.0266 | 0.0266 | 0.0953 | 0.1959 | 0.2141 | 0.1145 | | | |
| IFN-γ + hueIF4A | 0.0401 | 0.0402 | 0.4967 | 0.1268 | 0.1348 | 0.2619 | 0.0104 | | |
| IFN-γ + eIF4AIII | 0.0369 | 0.0369 | 0.2633 | 0.1613 | 0.0859 | 0.1844 | 0.0719 | 0.0581 | |
| IFN-γ + Ded1 | 0.0169 | 0.007 | 0.1086 | 0.0488 | 0.0498 | 0.0497 | 0.0701 | 0.1177 | 0.1017 |

The results presented in FIGS. 7B and C show that the proteins yeIF4A, FAL1, hueIF4A, eIF4AIII and Ded1 induce the secretion of levels of IL-10 and of TNF-α significantly higher than those induced in the supernatants of unstimulated cultures of monocytes (p<0.05, cf. Table 2 below).

The proteins FAL1, hueIF4A, eIF4AIII and Ded1 induce the production of levels of IL-10 and of TNF-α comparable to those induced by the soluble or insoluble protein LeIF (p<0.05). However, the yeast protein yeIF4A induces the production of levels of IL-10 and of TNF-α significantly lower than those induced by the soluble or insoluble protein LeIF (p<0.05, Table 2).

Table 2 below shows the statistical analysis of the differences observed in the levels of IL-10 and of TNF-α in the culture supernatants of the monocytes stimulated with the recombinant proteins yeIF4A, FAL1, hueIF4A, eIF4AIII and Ded1. The p values determined by the paired t test are shown.

TABLE 2

| | | NS | LPS | LeIF | LeIF (S) | yeIF4A | FAL1 | hueIF4A | eIF4AIII |
|---|---|---|---|---|---|---|---|---|---|
| IL-10 | LPS | 0.0014 | | | | | | | |
| | LeIF | 0.0013 | 0.004 | | | | | | |
| | LeIF (S) | 0.0037 | 0.0121 | 0.1634 | | | | | |
| | yeIF4A | 0.0022 | 0.151 | 0.0049 | 0.0089 | | | | |
| | FAL1 | 0.0029 | 0.0227 | 0.1584 | 0.2619 | 0.0007 | | | |
| | hueIF4A | 0.0362 | 0.0682 | 0.09 | 0.1181 | 0.1152 | 0.2091 | | |
| | eIF4AIII | 0.0394 | 0.0168 | 0.6321 | 0.6096 | 0.0776 | 0.0559 | 0.0552 | |
| | Ded1 | 0.0229 | 0.5827 | 0.724 | 0.0888 | 0.8532 | 0.029 | 0.1484 | 0.1141 |
| TNF-α | LPS | 0.0062 | | | | | | | |
| | LeIF | 0.0063 | 0.0006 | | | | | | |
| | LeIF (S) | 0.005 | 0.0006 | | | | | | |
| | yeIF4A | 0.0049 | 0.3789 | 0.0442 | 0.0365 | | | | |
| | FAL1 | 0.0066 | 0.0927 | 0.4029 | 0.3803 | 0.0541 | | | |
| | hueIF4A | 0.0046 | 0.4905 | 0.3365 | 0.3523 | 0.1347 | 0.4965 | | |
| | eIF4AIII | 0.0288 | 0.2621 | 0.1469 | 0.063 | 0.1673 | 0.5381 | 0.335 | |
| | Ded1 | 0.016 | 0.7602 | 0.1256 | 0.132 | 0.0514 | 0.1768 | 0.2411 | 0.135 |

All these results show that the homologous proteins of the protein LeIF: yeast yeIF4A, FAL1 and Ded1 and mammalian hueIF4A and eIF4AIII, are also capable of inducing the production of cytokines IL-12p70, IL-10 and TNF-α by human monocytes in vitro.

III.3. The Activity Inducing the Production of Cytokines by the Various Recombinant Proteins is Proteinase K Sensitive and Polymixin B Resistant.

To verify absence of contaminants of bacterial origin (LPS) in the preparations of recombinant proteins used, the levels of IL-10 produced in cultures of monocytes stimulated by the recombinant protein or by LPS in the presence or absence of proteinase K (endopeptidase that degrades the proteins) or of polymixin B (which has the effect of blocking the action of LPS) were compared. The results presented in FIG. 8 show that proteinase K causes more than 50% inhibition of the production of IL-10 by the recombinant proteins, whereas polymixin B has no effect on this activity. In contrast to recombinant proteins, the IL-10 inducing activity of LPS is strongly inhibited by polymixin B, but is resistant to treatment with proteinase K (FIG. 8).

These results show that the various antigenic preparations used are not contaminated with bacterial endotoxins.

### EXAMPLE 2

### Identification of the Fragments of the Protein LeIF that Induce the Production in Vitro of the Cytokines IL-12p70, IL-10 and TNF-Alpha

With the aim of identifying the minimum region responsible for the induction of cytokines within the protein LeIF, several fragments of this protein were produced. On the basis that the regions NH₂ of LeIF of L. braziliensis and L. major have been described as being responsible for the immunomodulating activity of these proteins, whereas a loss of activity was observed for the central regions and COOH (Probst et al., 1997; Skeiky et al., 1998), the NH₂ (1-226; SEQ ID NO: 18), central (129-261; SEQ ID NO: 23) and COOH (196-403; SEQ ID NO: 24) parts of the protein LeIF of L. infantum were constructed. Two other fragments within region NH₂ were also constructed: 1-195 (SEQ ID NO: 17) and 129-226 (SEQ ID NO: 22).

Isolation of mononuclear cells (PBMC), induction of secretion of cytokines and assay by sandwich ELISA were carried out in the same conditions as those described in Example 1. Briefly, monocytes purified from PBMCs of 6 healthy individuals were primed for 12 h at 37° C. by IFN-γ, then stimulated, either by the various proteins or fragments at final concentrations of 10 μg/ml or by LPS at 1 μg/ml, for 24 h for production of IL-12p70. For production of IL-10 and TNF-α, the monocytes were stimulated for 18 h with the various recombinant proteins or with LPS. The levels of IL-12, IL-10 and TNF-α are determined by sandwich ELISA.

1. Analysis of IL-12p70 Inducing Capacity

FIG. 9(A) shows the results of experiments carried out in five individuals. Table 3 below shows statistical analysis of the differences observed in the levels of IL-12p70 induced in the culture supernatants of the monocytes stimulated by the protein LeIF and its various insoluble fragments. The p values determined by the paired t test are shown.

TABLE 3
IL-12p70

| | | | IFN-γ + | IFN-γ + | IFN-γ + | IFN-γ + | IFN-γ + | IFN-γ + |
|---|---|---|---|---|---|---|---|---|
| | NS | IFN-γ | LPS | LeIF | NH2 | COOH | 129-261 | 1-195 |
| IFN-γ | 0.3132 | | | | | | | |
| IFN-γ + LPS | 0.0108 | 0.0108 | | | | | | |
| IFN-γ + LeIF | 0.0169 | 0.0169 | 0.1276 | | | | | |
| IFN-γ + NH2 | 0.0273 | 0.0272 | 0.1614 | 0.0144 | | | | |
| IFN-γ + COOH | 0.0251 | 0.0250 | 0.0787 | 0.0363 | 0.0606 | | | |
| IFN-γ + 129-261 | 0.0686 | 0.0685 | 0.3537 | 0.0193 | 0.0191 | 0.0085 | | |
| IFN-γ + 1-195 | 0.0284 | 0.0283 | 0.1387 | 0.0052 | 0.589 | 0.2398 | 0.0332 | |
| IFN-γ + 129-226 | 0.0575 | 0.0570 | 0.2529 | 0.0212 | 0.0398 | 0.0143 | 0.1690 | 0.1238 |

In contrast to the two fragments 129-261 and 129-226, the levels of IL-12p70 induced by stimulation with the fragments NH₂ (1-226) (3313.43±1375.7 pg/ml); (1-195) (3532±1485.8 pg/ml) and COOH (196-403) (4764.2±1935.5 pg/ml) are significantly higher than those produced by the monocytes that were not stimulated (24.78±10.76 pg/ml, p<0.05, Table 3) or stimulated with the recombinant proteins NH₂ (1-226), 1-195 or COOH (196-403) alone (202.91±94.55, 205.93±89.43 and 267.19±101.87 pg/ml, respectively; p<0.05) or IFN-γ alone (21.56±11.64 pg/ml) (p<0.05, cf. Table 3).

The levels of IL-12p70 induced in the culture supernatants of monocytes activated by IFN-γ and stimulated by the whole protein LeIF (5375.63±1683 pg/ml) are significantly higher than those induced by all the fragments (p<0.05, cf. Table 3) and comparable to those induced by LPS (2678.3±783.51 pg/ml, p>0.05, cf. Table 3).

In contrast to what was reported in the literature (Probst et al., 1997), the COOH part (195-403) of the protein LeIF of the species L. infantum induces the production of IL-12p70 (4764.2±1935.5 pg/ml) by monocytes activated by IFN-γ. These levels of IL-12p70 are comparable to those induced by the NH₂ part (1-226) (3313.43±1375.7 pg/ml, p>0.05, Table 3) or the 1-195 fragment (3532±1485.8 pg/ml, p>0.05, Table 3). Since the 129-261 fragment induces low levels of IL-12p70, the inducing activity within the COOH part (196-403) might be localized in the minimum region corresponding to positions 261 to 403.

Within the NH₂ region, it was shown that, in contrast to the results obtained with the 129-226 fragment, the 1-195 fragment induces significant levels of IL-12p70, which suggests that within this NH₂ region the inducing activity is localized at the level of the 1-129 fragment.

2. Analysis of IL-10 Inducing Capacity

The results illustrated in FIG. 9(B) show that all the fragments induce the secretion of levels of IL-10 that are significantly higher than those induced in the culture supernatants of the monocytes that were not stimulated (27.53±24.75; p<0.05, cf. Table 4). The difference between the levels of IL-10 induced by the NH₂ part or the 1-195 fragment and the COOH part (196-403), is not significant (p>0.05, cf. Table 4 below).

The NH2 (1-226) and 1-195 fragments induce the secretion of levels of IL-10 that are significantly higher than those induced by stimulation by LPS (1361.43±261.9 pg/ml for NH2 and 1331.17±228.35 pg/ml for 1-195; p<0.05, cf. Table 4) and by fragments 129-261 (960.26±229.99 pg/ml, p<0.05, Table 10) and 129-226 (631.09±134.59 pg/ml, p<0.05, cf. Table 4).

However, the whole protein LeIF is still more effective than all the fragments analyzed as well as LPS in induction of the production of IL-10 (2329.94±322.02 pg/ml for LeIF and 664.8±106.71 pg/ml for LPS; p=0.0032, cf. Table 4).

Table 4 below shows statistical analysis of the differences observed for the levels of production of IL-10 in the culture supernatants of the monocytes stimulated with the various recombinant proteins. The p values determined by the paired t test are shown.

TABLE 4

| | | | | NH2 | COOH | | |
|---|---|---|---|---|---|---|---|
| | NS | LPS | LeIF | (1-226) | (195-403) | 129-261 | 1-195 |
| LPS | 0.0014 | | | | | | |
| LeIF | 0.0009 | 0.0032 | | | | | |
| NH2 (1-226) | 0.0042 | 0.0447 | 0.0098 | | | | |
| COOH (195-403) | 0.072 | 0.4294 | 0.0139 | 0.0895 | | | |
| 129-261 | 0.0174 | 0.28 | 0.0029 | 0.0263 | 0.2569 | | |
| 1-195 | 0.0026 | 0.0356 | 0.0008 | 0.826 | 0.5313 | 0.0322 | |
| 129-226 | 0.0064 | 0.8171 | 0.0017 | 0.0041 | 0.4238 | 0.0254 | 0.0057 |

3. Analysis of TNF-α Inducing Capacity

TNF-α was assayed in the culture supernatants of monocytes purified from PBMCs from 6 healthy donors. FIG. 9(C) shows that the whole protein LeIF as well as all its fragments induce the production of levels of TNF-α significantly higher than those observed in the culture supernatants of the monocytes that were not stimulated (LeIF (9781.51±1535.1 pg/ml); NH₂ (1-226) (8430.47±1653 pg/ml); COOH (196-403) (9339.07±1334.3 pg/ml); 129-261 (5341.45±1281.1 pg/ml); 1-195 (8556.9±1856.3 pg/ml); 129-226 (5543.69±1185.7 pg/ml) and NS (136.64±54.5 pg/ml), p<0.05, cf. Table 5 below).

The fragments COOH (195-403), NH₂ (1-226) and 1-195 induce the secretion of levels of TNF-α comparable to those induced by the protein LeIF (p>0.05, Table 5). Once again, the 129-261 and 129-226 fragments induce the lowest levels of TNF-α (5341.45±1281.1 and 5543.69±1185.7 pg/ml, respectively).

Table 5 shows statistical analysis of the differences observed in the levels of TNF-α in the culture supernatants of the monocytes stimulated with the soluble protein LeIF and its various domains. The p values determined by the paired t test are shown.

TABLE 5

| | | | | NH2 | COOH | | |
|---|---|---|---|---|---|---|---|
| | NS | LPS | LeIF | (1-226) | (195-403) | 129-261 | 1-195 |
| LPS | 0.0062 | | | | | | |
| LeIF | 0.0015 | 0.0008 | | | | | |
| NH2 (1-226) | 0.0038 | 0.1423 | 0.1597 | | | | |
| COOH (196-403) | 0.0009 | 0.0521 | 0.4819 | 0.3622 | | | |
| 129-261 | 0.0138 | 0.3044 | 0.0213 | 0.0188 | 0.0126 | | |
| 1-195 | 0.006 | 0.1414 | 0.2958 | 0.8145 | 0.5755 | 0.0254 | |
| 129-226 | 0.0056 | 0.0838 | 0.0058 | 0.0057 | 0.013 | 0.3635 | 0.0122 |

### EXAMPLE 3

### Induction, by the Mutant Protein LeIFK76A, of the Secretion of the Cytokines IL-12p70, IL-10 and TNF-Alpha by Human Monocytes in Vitro

The effect of substitution in motif I, of the lysine in position 76 with alanine, on the cytokine inducing capacity of the protein LeIF was evaluated. This mutation cancels the ATPase activity of the protein LeIF (Barhoumi et al., 2006).

Isolation of the mononuclear cells (PBMC), induction of the secretion of cytokines and assay by sandwich ELISA were carried out in the same conditions as those described in Example 1. Briefly, the monocytes purified from the PBMCs from 3 individuals primed or not with IFN-γ for 12 h, are stimulated by the proteins LeIF (SEQ ID NO: 11) or LeIFK76A (SEQ ID NO: 27) at final concentrations of 10 μg/ml, or by LPS at 1 μg/ml. The culture supernatants are collected after incubation for 24 h for assay of IL-12p70 or 18 h for assay of IL-10 and TNF-α. The levels of the cytokines are determined by sandwich ELISA.
IL-12p70:

The results presented in FIG. 10(A) show that the protein LeIFK76A induces the production of high levels of IL-12p70 (2272.78±1257.5 pg/ml) by monocytes activated by IFN-γ. These levels are significantly higher than those induced in the culture supernatants of the monocytes that were not stimulated (29.69±24, 31 pg/ml) or were stimulated by the recombinant protein LeIFK76A alone (342.55±323.24 pg/ml) or IFN-γ alone (22.43±17.14 pg/ml; p<0.05, cf. Table 6 below).

They are comparable to those induced by the soluble recombinant protein LeIF (2564.46±1237.1 pg/ml).

Table 6 shows statistical analysis of the differences observed for the levels of production of IL-12p70 in the culture supernatants of the monocytes stimulated with the proteins LeIF and LeIFK76A. The p values determined by the paired t test are shown.

TABLE 6

| | NS | IFN-γ | IFN-γ + LPS | IFN-γ + LeIF |
|---|---|---|---|---|
| IFN-γ | 0.3132 | | | |
| IFN-γ + LPS | 0.0108 | 0.0108 | | |
| IFN-γ + LeIF(S) | 0.0193 | 0.0190 | 0.0468 | 0.1468 |
| IFN-γ + LeIFK76A | 0.0440 | 0.0434 | 0.1828 | 0.1387 |

IL-10 and TNF-α:

The results presented in FIGS. 10(B) and (C) show that the protein LeIFK76A induces the production of levels of IL-10 (1766.24±582.5 pg/ml) and of TNF-α (10303.36±1032.5 pg/ml) significantly higher than those induced in the culture supernatants of the monocytes that were not stimulated (27.53±24.75 pg/ml; p<0.05, cf. Table 7 below), but comparable to those induced by the soluble protein LeIF (2138.54±555.59 pg/ml for IL-10 and 11946.49±623.04 pg/ml for TNF-α, p>0.05, Table 7).

Table 7 shows statistical analysis of the differences observed in the levels of IL-10 and of TNF-α in the culture supernatants of the monocytes stimulated with the proteins LeIF and LeIFK76A. The p values determined by the paired t test are shown.

TABLE 7

| | | NS | LPS | LeIF (S) |
|---|---|---|---|---|
| IL-10 | LPS | 0.0014 | | |
| | LeIF(S) | 0.0037 | 0.0121 | |
| | LeIFK76A | 0.0094 | 0.1665 | 0.0672 |
| TNF-α | LPS | 0.0062 | | |
| | LeIF(S) | 0.005 | 0.0006 | |
| | LeIFK76A | 0.0085 | 0.2627 | 0.2307 |

All these results show that substitution, in motif I, of the lysine in position 76 with alanine, which cancels the ATPase activity of the protein LeIF, does not affect its activity for inducing the cytokines IL-12p70, IL-10 and TNF-α.

### EXAMPLE 4

### Induction, by the Proteins LeIFΔ25, D1+25, D1 and D2, of Secretion of the Cytokines IL-12p70, IL-10 and TNF-Alpha by Human Monocytes in Vitro

In order to characterize the cytokine inducing activity of the recombinant proteins LeIFΔ25 (protein LeIF of L. infantum after deletion of the first 25 amino-terminal residues that are the most divergent between LeIF and its yeast and mammalian homologs; SEQ ID NO: 21), D1 (25-237; SEQ ID NO: 20), D1+25 (1-237; SEQ ID NO: 19) which corresponds to the NH₂ part and D2 (237-403; SEQ ID NO: 25) which corresponds to the COOH part of the protein LeIF of L. infantum, monocytes purified from the PBMCs from five healthy individuals by these various recombinant proteins at final concentrations of 10 μg/ml were stimulated.

The stimulation conditions are identical to those described in Example 1. Briefly, the monocytes purified from the PBMCs from five healthy individuals, primed for 12 h with IFN-γ (3000 U/ml), or not, are stimulated by the soluble protein LeIF and its various domains at final concentrations of 10 μg/ml or by LPS at 1 μg/ml. The culture supernatants are collected after 24 h of incubation for assay of IL-12 or 18 h for assay of IL-10 and of TNF-α. The levels of IL-12, IL-10 and TNF-α are determined by sandwich ELISA.
IL-12:

The results presented in FIG. 11(A) show that the various recombinant proteins LeIFΔ25, D1+25, D1 and D2 induce levels of IL-12-p70 (2921.95±905.78; 2766.71±942.32; 2226.48±1188.2 and 1482.99±596.89 pg/ml, respectively) significantly higher than those induced in the culture supernatants of the monocytes that were not stimulated (34.17±17.77 pg/ml; p<0.05, cf. Table 8 below) or stimulated by IFN-γ alone (35.53±17.84 pg/ml, p<0.05, cf. Table 8) or by the protein alone (LeIFΔ25 (312.22±165.25), D1+25 (339.74±181.26), D1 (214.26±191.55) and D2 (243.28±132.6)).

It should be noted that deletion of the amino-terminal residues 25 does not affect the cytokine IL-12p70 inducing activity of the protein LeIF. In fact, the difference between the levels of IL-12p70 induced by the soluble protein LeIF (3805.9±1069.1 pg/ml) and the protein LeIFΔ25 (2921.95±905.78 pg/ml) is not significant (p>0.05, cf. Table 8). The domains D1+25 (1-237) and D1 induce the production of levels of IL-12p70 comparable to those induced by the protein LeIFΔ25 and significantly higher than those induced by the domain D2 (1482.99±596.89 pg/ml; p<0.05, Table 8).

The difference between the levels of IL-12p70 induced by the domain D1 and those induced by the domain D2 of the protein LeIF may be due to the difference in the number of DEAD-box motifs between the two domains. In fact, domain D1 contains 6 motifs (Q, Ia, Ib, I, II and III) and domain D2 contains the 3 motifs: IV, V and VI. This suggests that the DEAD-box motifs might play a role in the activity of inducing cytokine IL-12p70 by the protein LeIF.

Table 8 shows statistical analysis of the differences observed in the levels of IL-12p70 in the culture supernatants of the monocytes stimulated with the soluble protein LeIF and its various domains. The p values determined by the paired t test are shown.

TABLE 8

| | | | IFN-γ + | IFN-γ + | IFN-γ + | IFN-γ + | IFN-γ + |
|---|---|---|---|---|---|---|---|
| | NS | IFN-γ | LPS | LeIF(S) | LeIFΔ25 | D1 + 25 | D1 |
| IFN-γ | 0.3132 | | | | | | |
| IFN-γ + LPS | 0.0108 | 0.0108 | | | | | |
| IFN-γ + LeIF (s) | 0.0193 | 0.0190 | 0.0468 | | | | |
| IFN-γ + LeIFΔ25 | 0.0252 | 0.0248 | 0.1342 | 0.0827 | | | |
| IFN-γ + D1 + 25 | 0.0314 | 0.0310 | 0.1641 | 0.0471 | 0.1777 | | |
| IFN-γ + D1 | 0.0345 | 0.0341 | 0.1831 | 0.0425 | 0.1364 | 0.1077 | |
| IFN-γ + D2 | 0.0475 | 0.0465 | 0.3485 | 0.013 | 0.0098 | 0.0196 | 0.0290 |

IL-10:

The results presented in FIG. 11(B) show that the various recombinant proteins LeIFΔ25, D1+25 (1-237), D1 (25-237) and D2 (237-403) induce the secretion of levels of IL-10 (1951.15±408.4; 1477.88±381.92; 1408.91±269.75 and 1130.55±184.9 pg/ml) significantly higher than those induced in the culture supernatants of the monocytes that were not stimulated (32.14 pg/ml; p<0.05, cf. Table 9 below).

The protein LeIFΔ25 induces the production of levels of IL-10 (1951.15±408.4 pg/ml) comparable to those induced by the protein LeIF (2067.68±324.69 pg/ml, p=0.4515, cf. Table 9), by the domain D1+25 (1-237) (1477.88±381.92 pg/ml; p=0.0786, Table 9) or by the domain D1 (25-237) (1408.91±269.75 pg/ml; p=0.0786, cf. Table 9). The deletion of the 25 aminoterminal residues does not affect the IL-10 inducing activity of the protein LeIF.

Finally, it should be noted that the domain D2 (237-403) of the protein LeIF induces production of a level of IL-10 (1130.55±184.9 pg/ml) significantly lower than that induced by the protein LeIF (S) or LeIFΔ25 (p<0.05, cf. Table 9) and comparable to that induced by the domain D1+25 (1-237) or D1 (25-237) (p>0.05, cf. Table 9).

Table 9 shows statistical analysis of the differences observed in the levels of IL-10 in the culture supernatants of the monocytes stimulated with the soluble protein LeIF and its various domains. The p values determined by the paired t test are shown.

TABLE 9

| | | | | | D1 + 25 | D1 |
|---|---|---|---|---|---|---|
| | NS | LPS | LeIF (S) | LeIFΔ25 | (1-237) | (25-237) |
| LPS | 0.0014 | | | | | |
| LeIF (S) | 0.0037 | 0.0121 | | | | |
| LeIFΔ25 | 0.0104 | 0.0371 | 0.4515 | | | |
| D1 + 25(1-237) | 0.0197 | 0.114 | 0.0736 | 0.0781 | | |
| D1 (25-237) | 0.0075 | 0.2426 | 0.0436 | 0.0786 | 0.9861 | |
| D2 (237-403) | 0.0051 | 0.0785 | 0.0026 | 0.0272 | 0.2757 | 0.4896 |

TNF-α:

The results presented in FIG. 11(C) show that all the recombinant proteins LeIFΔ25, D1+25 (1-237), D1 (25-237) and D2 (237-403) induce the production of levels of TNF-α significantly higher than those induced in the culture supernatants of the monocytes that were not stimulated (8353.4±1827; 7612.07±1250.7; 8743.63±1419; 6104.9±1419 and 136.64±54.5 pg/ml for the proteins LeIFΔ25, D1+25 (1-237), D1 (25-237), D2 (237-403) and NS, respectively; p<0.05; cf. Table 10 below).

With the exception of the domain D2, the three other proteins LeIFΔ25, D1+25, D1 induce levels of TNF-α comparable to those induced by the soluble protein LeIF (9305.21±1656.3 pg/ml; p>0.05, cf. Table 10).

Table 10 shows statistical analysis of the differences observed in the levels of TNF-α in the culture supernatants of the monocytes stimulated with the soluble protein LeIF and its various domains. The p values determined by the paired t test are shown.

TABLE 10

| | NS | LPS | LeIF (S) | LeIFΔ25 | D1 + 25 | D1 |
|---|---|---|---|---|---|---|
| LPS | 0.0062 | | | | | |
| LeIF(S) | 0.005 | 0.0006 | | | | |
| LeIFΔ25 | 0.0104 | 0.0493 | 0.158 | | | |
| D1 + 25(1-237) | 0.0036 | 0.1012 | 0.0584 | 0.4538 | | |
| D1(25-237) | 0.0231 | 0.8136 | 0.1531 | 0.0961 | 0.5901 | |
| D2(237-403) | 0.0123 | 0.7107 | 0.0357 | 0.0927 | 0.0503 | 0.1943 |

All these results show that all the domains of the protein LeIF are endowed with activity for inducing IL-12p70, IL-10 and TNF-α. However, a significant drop in this activity is observed for the domain D2. Moreover, deletion of the first 25 aminoterminal residues, which are unique to the protein LeIF of Leishmania, does not seem to affect the activity of the whole protein.

### REFERENCES

Altmann et al., EMBO, 1995, 14, 3820-3827.

Badaro et al., Braz J Infect Dis, 2001,5, 223-232.

Banroques et al., Mol Cell Biol, 2008, 28, 3359-71.

Barhoumi et al., FEBS J, 2006, 273, 5086-5100.

Bates et al., EMBO J, 2005, 24, 543-553.

Bente et al., Proteomics, 2003, 3, 9, 1811-29.

Benz et al., Structure Fold Des, 1999, 7, 671-679.

Bizebard et al., Biochemistry, 2004, 43, 7857-7866.

Carmel and Matthews, RNA, 2004, 40, 66-74.

Caruthers et al., Proc Natl Acad Sci USA, 2000, 97, 13080-13085.

Caruthers and McKay, Curr Opin Struct Biol, 2002, 12, 123-133.

Chan et al., RNA, 2004, 10, 200-209.

Chang et al., Nucleic Acids Res, 1997,25, 5033-5040.

Chuang et al., Science, 1997, 275, 1468-1471.

Coler et al., Infect Immun, 2002, 70, 4215-4225.

Coler et al., Infect Immun, 2007, 75, 4648-4654.

Cordin et al., EMBO J, 2004, 23, 2478-2487.

Cordin et al., Gene, 2006, 367, 17-37.

de la Cruz et al., Proc Natl Acad Sci USA, 1997, 94, 10, 5201-6.

de la Cruz et al., Trends Biochem Sci, 1999, 24, 192-198.

Emery et al., Mol Microbiol, 2004, 52, 141-158.

Ferraiuolo et al., Proc Natl Acad Sci USA, 2004, 101, 4118-4123.

Flores-Rozas and Hurwitz, J Biol Chem, 1993, 268, 21372-21383.

Fuller-Pace et al., EMBO J, 1993, 12, 3619-3626.

Gillian and Svaren, J Biol Chem, 2004, 279, 9056-9063.

Gorbalenya et al., Nucleic Acids Res., 1989, 17, 12, 4713-30.

Gorbalenya and Koonin, Curr Opin Struct Biol, 1993, 3, 419-429.

Hall and Matson, Mol Microbiol, 1999, 34, 867-877.

He et al., World J Gastroenterol, 2008, 14, 4, 532-40

Hirling et al., Nature, 1989, 339, 562-564.

Hodge et al., EMBO J, 1999, 18, 5778-5788.

Huang and Liu, J Biol Chem, 2002, 277, 12810-12815.

Ilyina et al., J. Mol Evol., 1992, 34, 4, 351-7.

Iost et al., J Biol Chem, 1999, 274, 17677-17683.

Johnson and McKay, RNA, 1999, 5, 1526-1534.

Kadare and Haenni, J Virol, 1997, 71, 2583-2590.

Kim et al., Structure, 1998, 6, 89-100.

Korolev et al., Cell, 1997, 90, 635-647.

Kossen and Uhlenbeck, Nucleic Acids Res, 1999, 27, 3811-3820.

Kossen et al., J Mol Biol, 2002, 324, 625-636.

Kressler et al., Mol Cell Biol, 1997, 17, 7283-7294.

Kressler et al., Mol Cell Biol, 1999, 19, 12, 7897-912.

Lamm et al., Nucleic Acids Res, 1996, 24, 3739-3747.

Lenzi et al., J Transl Med., 2007, 5, 66.

Li et al., Mol Cell Biol, 1999, 19, 7336-7346.

Linder, Biol Cell, 2003, 95, 3-4, 157-167.

Linder et al., Nature, 1989, 337, 121-122.

Linder and Stutz, Curr Biol, 2002, 11, 961-963.

Lorsh and Herschlag, Biochemistry, 1998a, 37, 2180-2193.

Luking et al., Crit Rev Biochem Mol Biol, 1998, 33, 259-296.

Myler et al., Proc Natl Acad Sci USA, 1999, 96, 2902-2906.

Noueiry et al., Proc Natl Acad Sci USA, 2000, 97, 12985-12990.

Nugent et al., Mol Biochem Parasitol, 2004, 136, 51-62.

Palacios et al., Nature, 2004, 427, 753-757.

Patel and Picha, Annu Rev Biochemi, 2000, 69, 651-697.

Pause and Sonenberg, EMBO J, 1992, 11, 2643-2653.

Pause et al., Mol Cell Biol, 1993, 13, 6789-6798.

Probst et al., Eur J Immunol, 1997, 27, 2634-2642.

Reed and Hurt, Cell, 2002, 108, 523-531.

Rocak and Linder, Nat Rev Mol Cell Biol, 2004, 5, 232-241.

Rocak et al., Nucleic Acids Res, 2005, 33, 999-10009.

Rogers et al., J Biol Chem, 1999, 274, 12236-12244.

Rogers et al., J Biol Chem, 2001a, 276, 12598-12608.

Rogers et al., J Biol Chem, 2001b, 276, 30914-30922.

Rogers et al., Prog Nucleic Acid Res Mol Biol, 2002, 72, 307-331.

Rossler et al., Nucleic Acids Res, 2001, 29, 2088-2096.

Rozen et al., Mol Cell Biol, 1990, 10, 1134-1144.

Sabel et al., Surgery, 2007, 142, 749-760.

Salay et al., Clin Vaccine Immunol, 2007, 14, 1173-1181.

Schmitt et al., EMBO J, 1999, 18, 4332-4347.

Schmid and Linder, Mol Microbiol, 1992, 6, 283-291.

Schwer and Mezaros, EMBO J, 2000, 19, 6582-6591.

Shi et al., Proc Natl Acad Sci USA, 2004, 101, 17628-17633.

Shibuya et al., Nat Struct Mol Biol, 2004, 11, 346-351.

Shiratori et al., Cancer Sci., 2007, 98,1936-1942.

Shneider et al., J Biol Chem, 2004, 279, 8617-8626.

Silverman et al., Gene, 2003, 312, 1-16.

Singleton and Wigley, J Bacteriol, 2002, 184, 1819-1826.

Skeiky et al., J Exp Med, 1995, 181, 1527-1537.

Skeiky et al., J Immunol, 1998, 161, 6171-6179.

Son & Kim, Arch Pharm Res., 2007, 30, 8, 1047-50.

Sonenberg and Diver, Curr Opin Stuct Biol, 2003, 13, 56-63.

Staley and Gutherie, Mol Cell, 1999, 3, 55-64.

Story and Steitz, Nature, 1992, 55, 374-376.

Story et al., Proc Natl Acad Sci USA, 2001, 98, 1465-1470.

Strasser and Hurt, Nature, 2001, 413, 648-652.

Subramanya et al., Nature, 1996, 28, 379-383.

Svitkin et al., RNA, 2001, 7, 382-394.

Tanner, Cell Cycle, 2003, 2, 9-18.

Tanner et al., Mol Cell, 2003, 11, 127-138.

Tanner and Linder, Mol Cell, 2001, 8, 251-262.

Theis et al., EMBO J, 1999, 18, 6899-6907.

Tseng et al., EMBO J, 1998, 17, 2651-2662.

Tsu and Uhlenbeck, Biochemistry, 1998, 37, 16989-16996.

Tsu et al., RNA, 2001, 7, 702-709.

Tuteja and Tuteja, Eur J Biochem, 2004a, 271, 1835-1840.

Tuteja and Tuteja, Eur J Biochem, 2004b, 271, 1849-1863.

Valdez et al., Eur J Biochem, 1997, 250, 800-807.

Valdez, Eur J Biochem, 2000, 267, 21, 6395402.

Velankar et al., Cell, 1999, 97, 75-84.

Walker et al., EMBO J, 1982, 1, 945-951.

Weiss et al., Expert Opin Biol Ther, 2007, 7, 1705-1721.

Wilson et al., BMC Mol Biol, 2004, 5, 11.

Xu et al., EMBO J, 2004, 23, 376-385.

Yan et al., Mol Cell Biol, 2003, 23, 414-423.

Yao et al., Nat Struct Biol, 1997, 4, 463-467.

Ye et al., Biochim Biophys Ada, 2004, 1659, 1-18.

Zhao et al., EMBO J, 2002, 21, 1177-1187.

Zhao et al., Structure, 2004, 12, 1373-1381.

1. Use of a yeast or mammalian DEAD-box RNA helicase comprising in its central part, arranged from (1) to (9) from the N-terminal end to the C-terminal end, the nine motifs of amino acids defined by the following consensus motifs: (1) motif Q (SEQ ID NO: 1), (2) motif I (SEQ ID NO: 2), (3) (SEQ ID NO: 3), (4) motif Ib (SEQ ID NO: 4), (5) motif II (SEQ ID NO: 5), (6) motif III, (SEQ ID NO: 6), (7) motif IV (SEQ ID NO: 7), (8) motif V (SEQ ID NO: 8) and (9) motif VI (SEQ ID NO: 9), of wild type or having a mutation in said consensus motif I (SEQ ID NO: 2), or of a fragment of the latter comprising either said six consensus motifs Q, I, Ia, Ib, II and III but not said three consensus motifs IV, V and VI, or said three consensus motifs IV, V and VI but not said six motifs Q, I, Ia, Ib, II and III, for inducing the production in vitro or ex vivo of at least one cytokine by a peripheral blood mononuclear cell (PBMC) of a mammal.

2. The use as claimed in claim 1, characterized in that said yeast belongs to the genus Saccharomyces.

3. The use as claimed in claim 1 or claim 2, characterized in that said cytokine is selected from the group comprising IL-12, IL-10 and TNF-alpha.

4. The use as claimed in any one of claims 1 to 3, characterized in that said DEAD-box RNA helicase of wild type is selected from the group comprising yeIF4A (SEQ ID NO: 12), FAL1 (SEQ ID NO: 13), Ded1 (SEQ ID NO: 14), hueIF4A (SEQ ID NO: 15) and eIF4AIII (SEQ ID NO: 16).

5. Use of a yeast or mammalian DEAD-box RNA helicase or of a fragment of the latter as defined in claim 1, 2 or 4 for preparing a medicinal product intended for treating or preventing infectious diseases or cancers.

6. The use as claimed in claim 5, characterized in that said medicinal product is useful as a vaccination adjuvant, for inducing an immune response of the Th1 type.

7. The use as claimed in claim 5 or claim 6, characterized in that said infectious diseases are parasitic infections, preferably infections induced by an intracellular parasite, notably leishmaniases.

8. The use as claimed in any one of claims 5 to 7, characterized in that said yeast belongs to the genus Saccharomyces.

9. The use as claimed in any one of claims 6 to 8, characterized in that said immune response consists of the production of at least one of the cytokines IL-12 and TNF-alpha.

10. A pharmaceutical composition comprising a yeast or mammalian DEAD-box RNA helicase or a fragment of the latter as defined in any one of the preceding claims and at least one pharmaceutically acceptable vehicle.

11. Products containing (i) a yeast or mammalian DEAD-box RNA helicase or a fragment of the latter, as defined in any one of the preceding claims and (ii) IFN-γ, for simultaneous, separate or sequential use in the treatment or prevention of cancer and of infectious diseases, preferably parasitic infections.

12. A fragment of the protein LeIF of Leishmania infantum, characterized in that it is selected from the fragments of the latter comprising either the six motifs Q (SEQ ID NO: 57), I (SEQ ID NO: 58), Ia (SEQ ID NO: 59), Ib (SEQ ID NO: 60), II (SEQ ID NO: 61) and III (SEQ ID NO: 62) but not the three motifs IV (SEQ ID NO: 63), V (SEQ ID NO: 64) and VI (SEQ ID NO: 65), or said three consensus motifs IV, V and VI but not said six motifs Q, I, Ia, Ib, II and III, and the fragments corresponding to the amino acids 1-195 (SEQ ID NO: 17), 1-237 (SEQ ID NO: 19), 25-237 (SEQ ID NO: 20), 26-403 (SEQ ID NO: 21), 129-226 (SEQ ID NO: 22), 237-403 (SEQ ID NO: 25) or 261-403 (SEQ ID NO: 26) of said protein LeIF.

13. The fragment as claimed in claim 12 or protein LeIF of Leishmania infantum of wild type or having a substitution of the lysine (K) in position 76 with an alanine (A) for use as a medicinal product.

14. The fragment or protein as claimed in claim 13, characterized in that the medicinal product is intended for treating or preventing infectious diseases or cancers.

15. The fragment or protein as claimed in claim 14, characterized in that the medicinal product is useful as a vaccination adjuvant for inducing an immune response of the Th1 type.

16. Products containing (i) a fragment as claimed in claim 12 or a protein as defined in claim 13 and (ii) IFN-γ, for simultaneous, separate or sequential use in the treatment or prevention of cancer and of infectious diseases, preferably parasitic infections.

17. Use of a fragment as claimed in claim 12 or of a protein as defined in claim 13 for inducing the production in vitro or ex vivo of IL-12 by a peripheral blood mononuclear cell (PBMC) of a mammal.
